(19) United States
(12) Patent Application Publication
(10) Pub. No.: US 2022/0039737 A1
(43) Pub. Date: Feb. 10, 2022

(54) ELECTROCHEMICAL BIOSENSING OF CANCER-INVOLVED LYMPH NODES

(71) Applicants: Mohammad Abdolahad, Tehran (IR); Ashkan Zandi, Tehran (IR); Zahra Davari-shamsabadi, Tehran (IR); Fatemeh Zahra Shojaeian Zanjani, Tehran (IR); Fereshteh Abbasvandi, Tehran (IR); Seyed Mohamad Sadegh Mousavi-kiasary, Tehran (IR); Afsoon Zandi, Tehran (IR); Ali Gilani, Tehran (IR); Yasin Kordehlachin, Tehran (IR)

(72) Inventors: Mohammad Abdolahad, Tehran (IR); Ashkan Zandi, Tehran (IR); Zahra Davari-shamsabadi, Tehran (IR); Fatemeh Zahra Shojaeian Zanjani, Tehran (IR); Fereshteh Abbasvandi, Tehran (IR); Seyed Mohamad Sadegh Mousavi-kiasary, Tehran (IR); Afsoon Zandi, Tehran (IR); Ali Gilani, Tehran (IR); Yasin Kordehlachin, Tehran (IR)

(73) Assignee: Nano Hesgarsazan Salamat Arya, Tehran (IR)

(21) Appl. No.: 17/508,996

(22) Filed: Oct. 24, 2021

Related U.S. Application Data
(60) Provisional application No. 63/105,218, filed on Oct. 24, 2020.

(51) Int. Cl. A61B 5/00; A61B 5/1468; A61B 5/0538; A61B 5/25

(52) U.S. Cl. CPC A61B 5/418; A61B 5/6848; A61B 5/25; A61B 5/0075; A61B 5/0538; A61B 5/1468

(57) ABSTRACT

A method for detecting cancerous status of a suspected lymph node (LN) to be cancerous. The method includes measuring fatty acid oxidation (FAO) in the suspected LN by measuring a charge transfer resistance (R_{CT}) associated with the suspected LN, and detecting, utilizing one or more processors, a cancerous status of the suspected LN. Detecting the cancerous status of the suspected LN includes detecting the suspected LN is cancer involved if the measured R_{CT} is less than a reference R_{CT} value, or detecting the suspected LN is healthy if the measured R_{CT} is more than the reference R_{CT} value.

### CROSS-REFERENCE TO RELATED APPLICATION

This application claims the benefit of priority from pending U.S. Provisional Patent Application Ser. No. 63/105,218 filed on Oct. 24, 2020, and entitled “A REAL-TIME INTRAOPERATIVE APPROACH TO DIAGNOSE METASTATIC LYMPH NODES”, which is incorporated herein by reference in its entirety.

### TECHNICAL FIELD

The present disclosure generally relates to cancer diagnosis, and particularly, to diagnosis of cancer involved lymph nodes by measuring lipid contents in a lymph node, which may be suspected to be cancerous, utilizing an electrochemical impedance spectroscopy (EIS) response recorded from the suspected lymph node.

### BACKGROUND

Lymph nodes (LNs) are parts of lymphatic system responsible for body immunity. There are hundreds of lymph nodes spread all over body linked together by lymphatic vessels (LV) and lymphatic fluid (LF). Continuous circulation of LF through LV makes LNs susceptible to be invaded at beginning of cancer invasion, and each involved LN may work as a site for tumor spreading. Hence, early detection of involved LNs, e.g., involved sentinel LNs (SLNs) is vital for managing therapeutic protocols.

On the other hand, most of the current diagnostic clinical approaches for detecting cancer involved LNs are based on dissecting a suspicious lymph node (LN) and evaluating the dissected LN. However, LN dissection has many side effects, such as causing body inflation and causing immune system defects because LN dissection may cause removing healthy LNs from a person's body. Lymph nodes play a key role in immune system; basically, lymph nodes filter foreign and undesirable substances such as infections, dead cells, cancer cells, etc. Hence, removing more lymph nodes causes greater damage to immune system. So, in-vivo approaches that do not require LN dissection from a patient's body are preferred to avoid dissection of cancer free LNs from body.

Hence, there is a need for a highly accurate device and method for diagnosing cancer involved LNs in early stages of cancer progression. Additionally, there is a need for a device and method for intraoperative in-vivo detection as well as in-vitro diagnosis applications of cancerous LNs that should be highly precise, fast, simple, and biocompatible with human body without causing any side effects.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for detecting cancerous status of a suspected lymph node (LN) to be cancerous. The method may include forming a lipid detection probe (LDP) by forming three lipid sensitive parts at three respective distal ends of three hollow needle electrodes by laser-assisted welding of a layer of carbon nanotubes (CNTs) onto surface of the respective distal ends of three platinum hollow needles, connecting a proximal end of each respective hollow needle electrode to an electrochemical stimulator-analyzer device, inserting the three lipid sensitive parts into the suspected LN, injecting a biocompatible electrolyte solution into the LN through a respective proximal end of at least one hollow needle electrode of the three hollow needle electrodes, measuring fatty acid oxidation (FAO) in the suspected LN by measuring a charge transfer resistance (R_{CT}) associated with the suspected LN, and detecting, utilizing one or more processors, a cancerous status of the suspected LN.

In an exemplary implementation, measuring FAO in the suspected LN by measuring the R_{CT} associated with the suspected LN may include recording an electrochemical impedance spectroscopy (EIS) from the suspected LN utilizing the LDP and the electrochemical stimulator-analyzer device where the recorded EIS may include a pseudo-semicircular curve and calculating R_{CT} of the recorded EIS.

In an exemplary implementation, calculating the R_{CT} of the recorded EIS may include forming a semicircle curve by complementing the pseudo-semicircular curve and measuring a diameter of the formed semicircle curve. In an exemplary implementation, calculating the R_{CT} of the recorded EIS may be done utilizing one or more processors.

In an exemplary implementation, detecting the cancerous status of the suspected LN may include detecting the suspected LN being cancer involved responsive to the measured R_{CT} being less than 110 kΩ, or detecting the suspected LN being healthy responsive to the measured R_{CT} being more than 110 kΩ.

In another general aspect, the present disclosure describes an exemplary method for detecting cancerous status of a suspected LN to be cancerous. The method may include measuring FAO in the suspected LN by measuring a R_{CT} associated with the suspected LN and detecting, utilizing one or more processors, a cancerous status of the suspected LN. In an exemplary implementation, detecting the cancerous status of the suspected LN may include one of detecting the suspected LN being cancer involved responsive to the measured R_{CT} being less than a reference R_{CT} value and detecting the suspected LN being healthy responsive to the measured R_{CT} being more than the reference R_{CT} value. In an exemplary implementation, measuring FAO in the suspected LN and detecting the cancerous status of the suspected LN may be done in a time period of less than one minute.

In an exemplary implementation, detecting the cancerous status of the suspected LN may include comparing, utilizing one or more processors, the measured R_{CT} with a reference RCT value of 110 kΩ, and detecting, utilizing one or more processors, one of situations in which the suspected LN is cancer involved if the measured R_{CT} is less than 110 kΩ or the suspected LN is healthy if the measured R_{CT} is less than 110 kΩ.

In an exemplary implementation, the method may further include generating the reference R_{CT} value. In an exemplary implementation, generating the reference R_{CT} value may include measuring a first set of R_{CT} values associated with a plurality of healthy lymph nodes (LNs), measuring a second set of R_{CT} values associated with a plurality of cancer involved LNs, and determining the reference R_{CT} value by determining a R_{CT} value at a border line magnitude between the first set of R_{CT} values and the second set of R_{CT} values.

In an exemplary implementation, measuring each of R_{CT} values associated with each LN of the plurality of cancer involved LNs, the plurality of healthy LNs, and the suspected LN may include inserting three lipid sensitive parts of three respective electrodes of a lipid detection probe (LDP) into a LN, increasing electrical conductivity inside the LN by injecting a biocompatible electrolyte solution into the LN, recording an electrochemical impedance spectroscopy (EIS) including a pseudo-semicircular curve from the LN utilizing the LDP, and calculating R_{CT} of the recorded EIS by measuring a diameter of a semicircle associated with the pseudo-semicircular curve.

In an exemplary embodiment, the LN may include one of the suspected LN, a LN of the plurality of cancer involved LNs, and a LN of the plurality of healthy LNs. In an exemplary embodiment, each respective electrode ma include a hollow needle electrode and each lipid sensitive part may include a distal end of each respective hollow needle electrode coated with a layer of lipophilic electrically conductive nanostructures. In an exemplary embodiment, the layer of lipophilic electrically conductive nanostructures may include a layer of carbon nanotubes (CNTs).

In an exemplary implementation, inserting three lipid sensitive parts of three respective electrodes of the LDP into the LN may include inserting each respective distal end of each electrode of the three electrodes into at least one of a LN in a living body and a dissected LN from a living body.

In an exemplary implementation, recording the EIS from the LN may include connecting the LDP to an electrochemical stimulator-analyzer device, applying an AC voltage between 5 mV and 10 mV by sweeping a frequency range including a plurality of frequency values between 0.01 Hz and 100 kHz, measuring a set of electrical impedance of the LN respective to the swept frequency range, and plotting a respective set of imaginary part of impedance (Z″ (Ω)) of the set of electrical impedance versus a respective set of real part of impedance (Z′ (Ω)) of the set of electrical impedance.

In an exemplary implementation, calculating the R_{CT} of the recorded EIS may include measuring a first intersection point of the pseudo-semicircular-shaped curve with Z′ (Ω) axis, generating a second intersection point between the pseudo-semicircular-shaped curve and Z′ (Ω) axis by adding a complementary sector to the pseudo-semicircular-shaped curve to form a semicircle, and measuring a distance between the first intersection point and the second intersection point.

In an exemplary implementation, injecting the biocompatible electrolyte solution into the LN may include injecting the biocompatible electrolyte solution through at least one of the three electrodes of the LDP by injecting the biocompatible electrolyte solution into a respective proximal end of the at least one of the three electrodes utilizing a syringe. In an exemplary implementation, injecting the biocompatible electrolyte solution into the LN may include injecting a biocompatible and electrically conductive solution of metal ions into the LN. In an exemplary implementation, injecting the biocompatible electrolyte solution into the LN may include injecting a solution of iron ions into the LN, where the solution of iron ions may include a colloidal solution of ferric carboxymaltose complex.

In an exemplary implementation, the method may further include fabricating the LDP. In an exemplary implementation, fabricating the LDP may include forming the three hollow needle electrodes with the three respective lipid sensitive parts, connecting respective first ends of three electrical connector lines to respective proximal ends of the three hollow needle electrodes, placing the three hollow needle electrodes with the respective electrical connector lines inside a handle, and forming an opening at a location of the handle above a location of respective proximal ends of the three hollow needle electrodes.

In an exemplary embodiment, a respective second end of each electrical connector line may be configured to be connected to an electrochemical stimulator-analyzer device. In an exemplary embodiment, the handle may include a hollow cylinder with a bottom surface at a first end of the hollow cylinder. In an exemplary embodiment, the handle may be configured to facilitate inserting the three hollow needle electrodes into the LN. In an exemplary embodiment, the respective distal end of each hollow needle electrode may be placed outside the bottom surface. In an exemplary embodiment, the opening may be configured to inject the biocompatible electrolyte solution there through into at least one of the three hollow needle electrodes.

In an exemplary implementation, forming the three hollow needle electrodes with the three respective lipid sensitive parts may include forming a bevel-shaped tip at a respective distal end of each of the three hollow needle electrodes and forming a layer of CNTs on the respective distal end of each of the three hollow needle electrodes. In an exemplary embodiment, the bevel-shaped tip may be configured to facilitate a non-invasive insertion of each of the three hollow needle electrodes into a LN. In an exemplary embodiment, the three hollow needle electrodes may include a set of electrochemical electrodes including a working electrode, a counter electrode, and a reference electrode.

In an exemplary implementation, forming the layer of CNTs on the respective distal end of each of the three hollow needle electrodes may include growing a layer of CNTs on each respective distal end of each of the three hollow needle electrodes and welding the grown layer of CNTs to the respective distal end of each of the three hollow needle electrodes utilizing a laser welding process. In an exemplary implementation, growing the layer of

CNTs on each respective distal end of each of the three hollow needle electrodes may include preparing a solution by dispersing CNTs in a mixture of ethanol and deionized water, immersing respective distal end of each of the three hollow needle electrodes in the solution of dispersed CNTs, and sonicating the solution of dispersed CNTs. In an exemplary implementation, welding the grown layer of CNTs to the respective distal end of each of the three hollow needle electrodes may include placing the three hollow needle electrodes with grown CNTs on the respective distal ends in a sealed container, filling the sealed container with a noble gas, and irradiating continuous-wave laser with a wavelength of 1024 nm to the three hollow needle electrodes with grown CNTs on the respective distal ends in the presence of the noble gas.

In an exemplary implementation, forming the three hollow needle electrodes with the three respective lipid sensitive parts may further include electrically insulating parts of surface of each hollow needle electrode by covering a layer of an electrical insulating material around the surface of each hollow needle electrode except a surface of the respective distal end of each hollow needle electrode.

In an exemplary implementation, placing the three hollow needle electrodes with the respective electrical connector lines inside the handle may include forming three openings with a triangular pattern at the bottom surface and placing the three hollow needle electrodes with the respective electrical connector lines inside the handle by passing each of the three hollow needle electrodes through a respective opening of the formed three openings. In an exemplary embodiment, three respective lipid sensitive parts may be placed outside the bottom surface. In an exemplary embodiment, each two openings may be apart from each other by a distance between 1 mm and 5 mm.

In an exemplary embodiment, the respective proximal end of each of the three hollow needle electrodes may be placed inside the handle. In an exemplary embodiment, a second end of each respective electrical connector line of the three electrical connector lines may be placed outside a second end of the handle.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A shows exemplary method for detecting cancerous status of a suspected lymph node (LN) to be cancerous, consistent with one or more exemplary embodiments of the present disclosure.

FIG. 1B shows a further implementation of an exemplary method for detecting cancerous status of a suspected LN to be cancerous, consistent with one or more exemplary embodiments of the present disclosure.

FIG. 2 shows an exemplary implementation of an exemplary method for measuring fatty acid oxidation (FAO) in a LN via measuring a charge transfer resistance (R_{CT}) of the LN, consistent with one or more exemplary embodiments of the present disclosure.

FIG. 3A shows a schematic view of an exemplary lipid detection probe (LDP), consistent with one or more exemplary embodiments of the present disclosure.

FIG. 3B shows a schematic view of exemplary laser-welded multi-walled carbon nanotubes (MWCNTs) onto an exemplary surface of an exemplary hollow needle electrode of three hollow needle electrodes, consistent with one or more exemplary embodiments of the present disclosure.

FIG. 4 shows an exemplary process for fabricating an exemplary LDP, consistent with one or more exemplary embodiments of the present disclosure.

FIG. 5 shows an exemplary schematic implementation of in-vivo inserting exemplary three lipid sensitive parts of three respective electrodes of an exemplary LDP into an exemplary LN, consistent with one or more exemplary embodiments of the present disclosure.

FIG. 6 shows a schematic view of an exemplary electrochemical impedance spectroscopy (EIS) plot recorded from an exemplary LN, consistent with one or more exemplary embodiments of the present disclosure.

FIG. 7A shows scanning electron microscopy (SEM) images of exemplary arrays of VAMWCNTs attached on an exemplary electrode of an exemplary prepared LDP and a magnified image of an exemplary attached VAMWCNTs array, consistent with one or more exemplary embodiments of the present disclosure.

FIG. 7B shows field emission-scanning electron microscopy (FE-SEM) images of exemplary array of VAMWCNTs grown on exemplary three-integrated electrodes of an exemplary prepared LDP, consistent with one or more exemplary embodiments of the present disclosure.

FIG. 8 shows contact angles of an exemplary bare platinum electrode and an exemplary MWCNT welded platinum electrode, consistent with one or more exemplary embodiments of the present disclosure.

FIG. 9A shows residue footprint after penetration of an exemplary prepared CNT welded platinum electrode compared to an exemplary platinum electrode with grown CNTs thereon, consistent with one or more exemplary embodiments of the present disclosure.

FIG. 9B shows FE-SEM images of an exemplary penetrated platinum electrode with grown CNTs thereon before and after penetration to an exemplary animal tissue in comparison with penetrated CNT welded platinum electrode before and after penetration to an exemplary animal tissue, consistent with one or more exemplary embodiments of the present disclosure.

FIG. 10 shows EIS analysis results of exemplary bare platinum electrodes versus exemplary MWCNT welded platinum electrodes, consistent with one or more exemplary embodiments of the present disclosure.

FIG. 11 shows EIS recorded diagrams of rabbit right and left popliteal LNs with and without injecting electrically conductive carrier solution, consistent with one or more exemplary embodiments of the present disclosure.

FIG. 12A shows EIS recorded diagrams of right popliteal lymph nodes of an exemplary rabbit for an exemplary LN with an injected lipidic solution, an exemplary normal lymph node, and an exemplary lymph node with injected tumescent solution and an inset diagram of respective R_{CT} of each EIS, consistent with one or more exemplary embodiments of the present disclosure.

FIG. 12B shows permanent and frozen Hematoxylin and eosin (H&E) histopathological staining results of exemplary normal and tumescent injected popliteal LNs of rabbit, consistent with one or more exemplary embodiments of the present disclosure.

FIG. 13 shows EIS recorded responses for a plurality of exemplary sentinel LNs of tested patients, consistent with one or more exemplary embodiments of the present disclosure.

FIG. 14 shows comparative EIS recorded responses, pathological analyzes results, and optical images for an exemplary cancer involved LN No. 1 of patient No. 7 and an exemplary normal LN No. 3 of patient No. 17 of Table 1, consistent with one or more exemplary embodiments of the present disclosure.

FIG. 15 shows R_{CT} values versus LNs' involvement percentage graph representing a relation between EIS results and cancer progression in exemplary LNs, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings. The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

In case of spreading a metastatic cancer throughout a patient' body, cancer cells may invade lymph nodes (LNs), for example, sentinel lymph nodes (SLNs). Cancer cells may invade LNs by migrating from a cancerous organ or tissue to a LN through circulatory system and/or lymphatic system. Herein, a “cancer involved lymph node (LN)” may refer to a LN which may be invaded by cancer cells by migrating cancer cells to the LN. Invasion of cancer cells into LNs causes changes in metabolism of cancer cells within LNs respective to cancer cells' metabolism in other organs/tissues. Specifically, cancer cells' metabolism may be hypoxia glycolysis in a cancerous tissue/organ, whereas cancer cells' metabolism may change to fatty acid oxidation (FAO) when they invade LNs since LNs are resources of lipids. Such metabolism change may cause consumption of lipid contents within an exemplary cancer involved LN. In other words, levels of fatty acid (FA) in normal LNs may be higher than those involved by malignant cells. Specifically, a metabolism change of cancer cells migrated to LNs from glycolysis toward FAO may cause lipid consumption by cancer cells within LNs, resulting in a depletion of LNs' tissue from lipid. Hence, herein an exemplary probe, method, and system based on tracing lipidic content variations in LNs is disclosed for cancer diagnosis within LNs and detection of cancer involved LNs.

Herein, an exemplary device and methods are disclosed for diagnosing cancer involved LNs via tracing fatty acid oxidation as a distinct metabolism of malignant cells that have invaded lymph nodes (LNs). Exemplary device and method are disclosed for intraoperative in-vivo detection as well as in-vitro diagnosis of cancer involved LNs; therefore, exemplary device and method may be capable of detecting a suspicious LN is either a cancer involved LN or a cancer free LN via a real-time, simple, and biocompatible with human body approach based on monitoring and/or measuring a lipid content within an exemplary lymph node.

In an exemplary embodiment, an exemplary method for distinguishing cancer involved LNs from normal (healthy) LNs is disclosed. In an exemplary embodiment, an exemplary method may include a bio-sensing method based on measuring levels of fatty acid content in LNs utilizing analysis of an electrochemical impedance spectroscopy (EIS) recorded response of an exemplary LN suspected to be cancerous utilizing an exemplary lipid detection probe (LDP). In an exemplary embodiment, an exemplary lipid detection probe (LDP) may be designed and fabricated for in-vivo and in-vitro FAO tracing in an exemplary LN via recording EIS of an exemplary LN utilizing exemplary fabricated LDP.

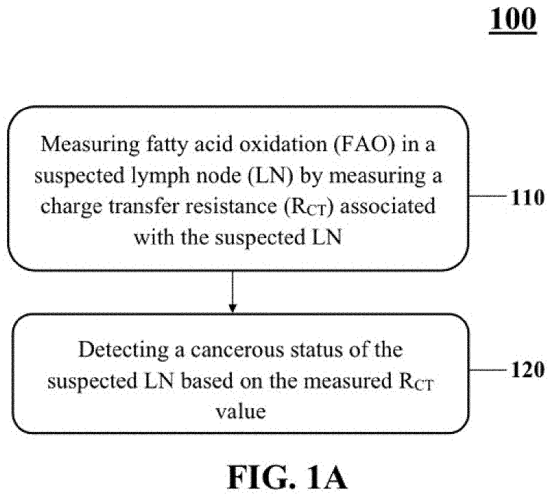

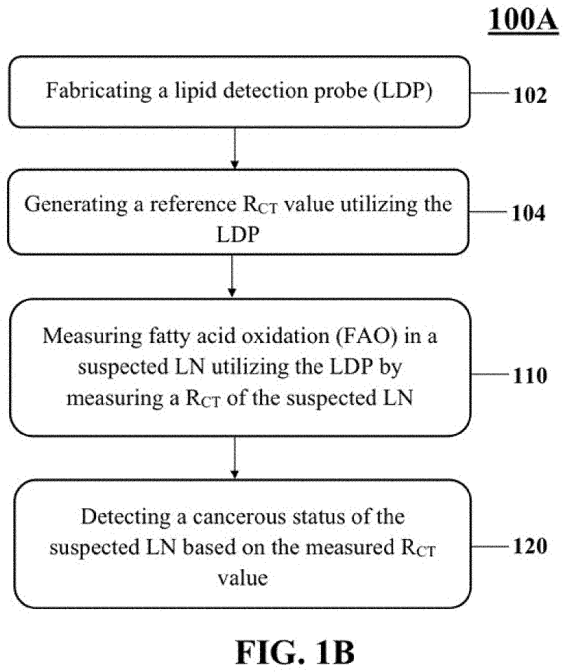

FIG. 1A shows exemplary method 100 for detecting cancerous status of a suspected lymph node (LN) to be cancerous, consistent with one or more exemplary embodiments of the present disclosure. Exemplary method 100 may include measuring fatty acid oxidation (FAO) in the suspected LN by measuring a charge transfer resistance (R_{CT}) associated with the LN (step 110) and detecting, utilizing one or more processors, a cancerous status of the suspected LN based on the measured R_{CT} value (step 120). In an exemplary implementation, exemplary method 100 may further include fabricating an exemplary lipid detection probe (LDP) for detecting cancerous status of a suspected LN as depicted in FIG. 1B. In an exemplary implementation referring to FIG. 1B, exemplary method 100A may further include fabricating an exemplary lipid detection probe (LDP) (step 102), generating a reference R_{CT} value utilizing the fabricated LDP (step 104), in addition to the steps of method 100, namely, measuring fatty acid oxidation (FAO) in a suspected lymph node (LN), utilizing the fabricated LDP, by measuring a R_{CT} of the suspected LN (step 110), and detecting, utilizing one or more processors, a cancerous status of the suspected LN based on the measured R_{CT} value (step 120).

In an exemplary implementation, exemplary methods 100 and 100A may be conducted in-vitro for detecting cancerous status of a suspected LN dissected from a living body. In an exemplary implementation, exemplary methods 100 and 100A may be conducted in-vivo for detecting cancerous status of a suspected LN within a living body. In an exemplary implementation, measuring FAO in an exemplary suspected LN (step 110) and detecting a cancerous status of the suspected LN (step 120) may be done in a time period of less than one minute, for example, in 10 seconds.

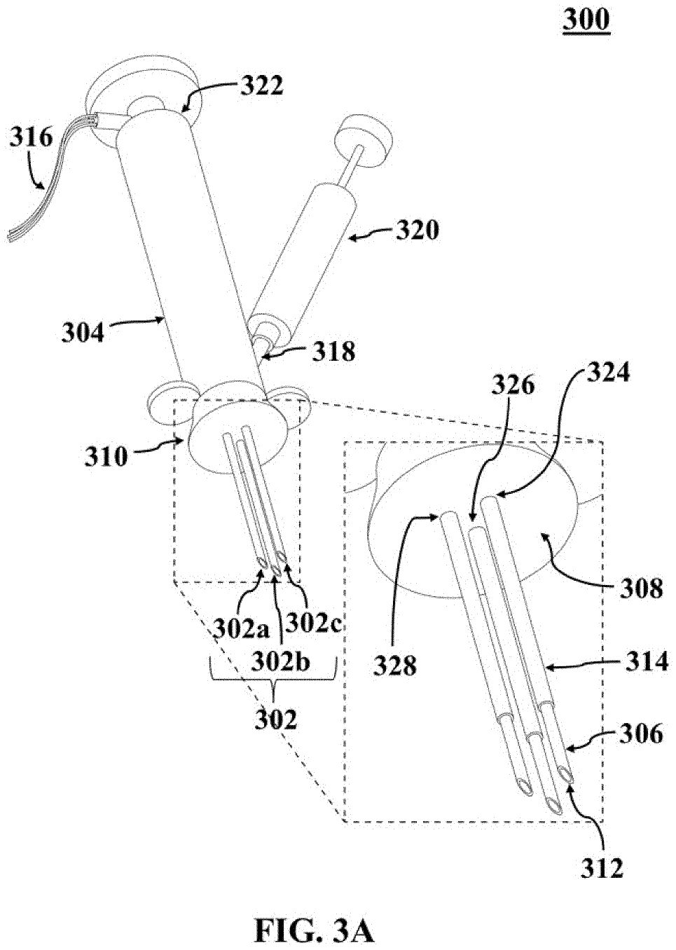

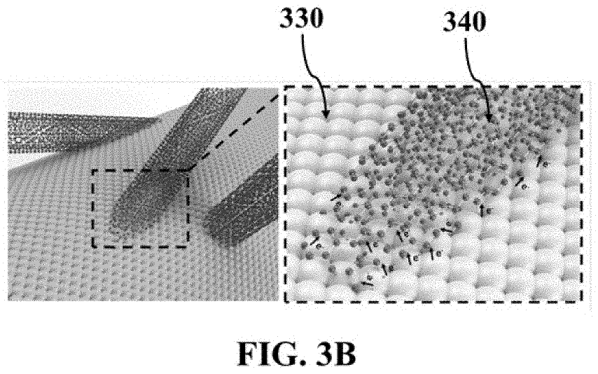

In detail, step 102 may include fabricating an exemplary lipid detection probe (LDP) that may be utilized via exemplary method 100 for detecting cancerous status of a suspected lymph node LN to be cancerous. FIG. 3A shows a schematic view of exemplary lipid detection probe (LDP) 300, consistent with one or more exemplary embodiments of the present disclosure. In an exemplary embodiment, LDP 300 may include three hollow needle electrodes 302 placed inside exemplary handle 304. In an exemplary embodiment, three hollow needle electrodes 302 may be configured to form a set of electrochemical electrodes for electrochemical measurements from a biological sample. In an exemplary embodiment, the set of electrochemical electrodes may include a working electrode (WE), a counter electrode (CE), and a reference electrode (RE). In an exemplary embodiment, three hollow needle electrodes 302 may form a set of working electrode 302a, counter electrode 302b, and reference electrode 302c for utilizing in electrochemical measurements of a sample, for example, recording an EIS response from an exemplary LN.

In an exemplary embodiment, exemplary handle 304 may include a hollow cylinder with bottom surface 308 at exemplary first end 310 of the hollow cylinder. In an exemplary embodiment, exemplary handle 304 may be configured to facilitate inserting three hollow needle electrodes 302 into an exemplary LN. In an exemplary embodiment, three hollow needle electrodes 302 may be placed inside exemplary handle 304, so that an exemplary lipid sensitive part 306 of each hollow needle electrodes of hollow needle electrodes 302 may be placed outside bottom surface 308.

In an exemplary embodiment, each lipid sensitive part 306 may include a distal portion of each respective hollow needle electrode of three hollow needle electrodes 302. Herein, an exemplary “distal portion” may refer to a length of about 5 mm to about 15 mm of total length of each of hollow needle electrodes 302a, 302b, and 302c from a respective distal end of each of hollow needle electrodes 302a, 302b, and 302c. In an exemplary embodiment, each lipid sensitive part 306 may include a distal portion of each respective hollow needle electrodes 302a, 302b, and 302c coated with a layer of a lipophilic electrically conductive material. In an exemplary embodiment, each lipid sensitive part 306 may include a distal portion of each respective hollow needle electrode coated with a layer of lipophilic electrically conductive nanostructures, for example, a layer of carbon nanotubes (CNTs). In an exemplary embodiment, the layer of CNTs may include a layer of at least one of single wall carbon nanotubes (SWCNTs), multi-walled carbon nanotubes (MWCNTs), vertically aligned multi-walled carbon nanotubes (VAMWCNTs), and combinations thereof. In an exemplary embodiment, the layer of CNTs, due to their hydrophobic nature and metallic conductivity, may be configured to provide an appropriate interfacial contact between lipids within an exemplary LN and hollow needle electrodes 302 while maintaining high electrical conductivity for impedance recording from exemplary LN to avoid parasitic resistance.

In an exemplary embodiment, each hollow needle electrode 302a, 302b, or 302c may include a biocompatible electrically conductive hollow needle. In an exemplary embodiment, each hollow needle electrode 302a, 302b, or 302c may include a needle made of at least one of gold, stainless steel, platinum, and combinations thereof In an exemplary embodiment, each hollow needle electrode 302a, 302b, or 302c may include a needle with a thickness between about 0.2 mm and about 1 cm. In an exemplary embodiment, each hollow needle electrode 302a, 302b, or 302c may include a needle with a thickness of about 0.5 mm.

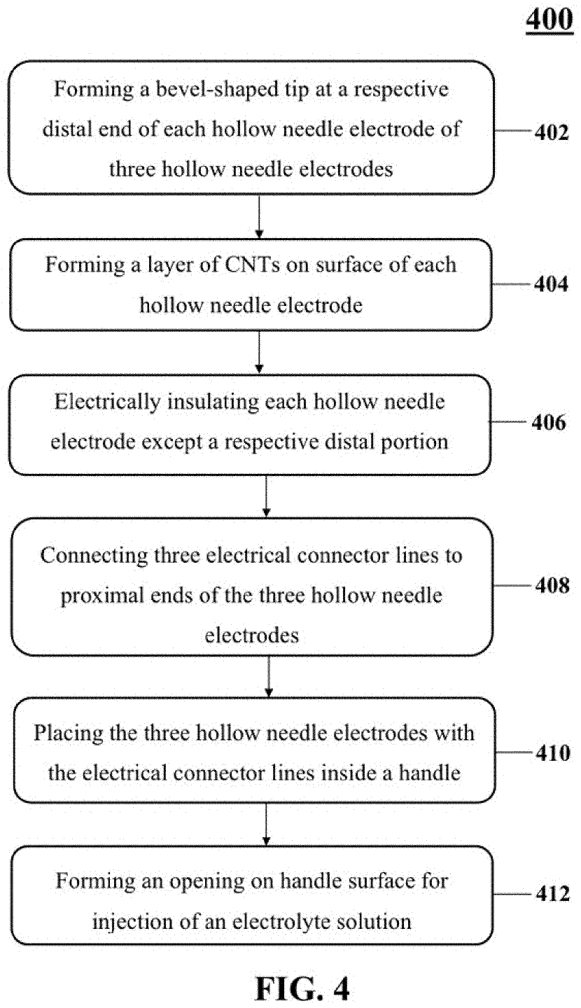

FIG. 4 shows an exemplary process 400 for fabricating exemplary LDP 300 (step 102), consistent with one or more exemplary embodiments of the present disclosure. In an exemplary implementation, fabricating exemplary lipid LDP 300 (step 102) may include forming a bevel-shaped tip 312 at a respective distal end of each of hollow needle electrodes 302a, 302b, and 302c (step 402), forming a layer of lipophilic electrically conductive nanostructures, for example, a layer of CNTs, on a respective surface of each of hollow needle electrodes 302a, 302b, and 302c (step 404), electrically insulating the respective surface of each of hollow needle electrodes 302a, 302b, and 302c except a respective distal portion of each of hollow needle electrodes 302a, 302b, and 302c (step 406), connecting respective first ends of three electrical connector lines 316 to respective proximal ends of hollow needle electrodes 302a, 302b, and 302c (step 408), placing three hollow needle electrodes 302a, 302b, and 302c with respective electrical connector lines 316 inside handle 304 (step 410), and forming an opening at a location of the handle above a location of respective proximal ends of the three hollow needle electrodes (step 412).

In an exemplary implementation, step 402 may include forming a bevel-shaped tip 312 at a respective distal end of each hollow needle electrode of three hollow needle electrodes 302; allowing for inserting and penetrating lipid sensitive part 306 into a LN without causing a rupture in the LN and surrounding tissue. In an exemplary implementation, forming bevel-shaped tip 312 at a respective distal end of each hollow needle electrode 302a, 302b, or 302c may further include thinning, utilizing wiring or cylinder formation methods, three cylindrical needles or three hollow wires to reach a thickness in a range between about 0.2 mm and 1 mm. In an exemplary implementation, forming bevel-shaped tip 312 at a respective distal end of each hollow needle electrode 302a, 302b, or 302c may include thinning three cylindrical needles or three hollow wires made of at least one of gold, stainless steel, platinum, and combinations thereof and forming a bevel-shaped tip 312 at a respective distal end of each of the three cylindrical needles or three hollow wires. In an exemplary implementation, each of the three cylindrical needles or three hollow wires may be thinned to reach a thickness size (or a diameter) at which deforming, displacing, bending, or breaking of hollow needle electrodes 302 may be prevented during penetration. In an exemplary implementation, each of the three cylindrical needles or three hollow wires may be thinned to reach a diameter of about 0.5 mm; allowing for preventing deforming, displacing, bending, or breaking of hollow needle electrodes 302 during penetration into an exemplary tissue or LN.

In an exemplary implementation, forming a bevel-shaped tip 312 at a respective distal end of each hollow needle electrode of three hollow needle electrodes 302 may be done via a mechanochemical method. In an exemplary implementation, forming bevel-shaped tip 312 at a respective distal end of each hollow needle electrode via an exemplary mechanochemical method may include forming bevel-shaped tip 312 via a process including both mechanical processes and chemical reactions. In an exemplary implementation, forming bevel-shaped tip 312 may include forming bevel-shaped tip 312 at a respective distal end of each hollow needle electrode 302a, 302b, or 302c using a mechanical process, including at least one of wedge metal cutting, metal shaping, metal polishing, and combinations thereof. Furthermore, forming bevel-shaped tip 312 may further include smoothing formed bevel-shaped tip 312 and removing microscale metal shavings from formed bevel-shaped tip 312 by applying a chemical process to formed bevel-shaped tip 312. In an exemplary implementation, an exemplary chemical process may include acid treatment, for example, using Nitric acid. In an exemplary implementation, a respective distal end of each hollow needle electrode 302a, 302b, or 302c may be treated by an acidic solution to form smooth and well sharped edges at the respective distal end.

Moreover, step 404 may include forming a layer of lipophilic electrically conductive nanostructures on a respective surface of each of hollow needle electrodes 302a, 302b, and 302c. In an exemplary implementation, forming the layer of lipophilic electrically conductive nanostructures on the respective surface of each of hollow needle electrodes 302a, 302b, and 302c may include forming a layer of CNTs on the respective surface of each of hollow needle electrodes 302a, 302b, and 302c. In an exemplary implementation, forming the layer of CNTs on the respective surface of each of hollow needle electrodes 302a, 302b, and 302c may include forming a layer of at least one of SWCNTs, MWCNTs, VAMWCNTs, and combinations thereof on the respective surface of each of hollow needle electrodes 302a, 302b, and 302c. In an exemplary embodiment, the layer of lipophilic electrically conductive nanostructures may include an array of VAMWCNTs with a length between about 2 μm and about 12 and a diameter between about 20 nm and about 75 nm for each VAMWCNT of the array of VAMWCNTs. In an exemplary embodiment, CNTs, for example, MWCNTs may have superhydrophobic properties as well as high electrical conductivity, so that the layer of CNTs may be configured to enhance an interaction of hollow needle electrodes 302a, 302b, and 302c with lipidic contents of an exemplary LN and increase electrical conductivity of hollow needle electrodes 302a, 302b, and 302c for signal transduction. For example, MWCNTs' contact resistance may be less than about 100Ω and MWCNTs' electrical conductivity may be about 40 S/cm to about 50 S/cm. Hydrophobicity of CNTs may be dependent on growth procedure and functionalization. However, hydrophobicity of CNTs may be measured by measuring a contact angle of CNTs with a non-lipidic solution. An exemplary measured contact angle of MWCNTs may be more than about 120° which shows a high hydrophobicity for MWCNTs.

In an exemplary implementation, forming the layer of CNTs on the respective surface of each of hollow needle electrodes 302a, 302b, and 302c may include forming the layer of CNTs on a portion of the respective surface of each of hollow needle electrodes 302a, 302b, and 302c. In an exemplary implementation, forming the layer of CNTs on the portion of the respective surface of each of hollow needle electrodes 302a, 302b, and 302c may include forming the layer of CNTs on a surface of a respective distal portion of each of hollow needle electrodes 302a, 302b, and 302c. In an exemplary implementation, forming the layer of CNTs on the portion of the respective surface of each of hollow needle electrodes 302a, 302b, and 302c may include forming the layer of CNTs on a portion surface of each of hollow needle electrodes 302a, 302b, and 302c corresponding to a length of a respective lipid sensitive part 306. In an exemplary implementation, forming the layer of CNTs on the portion of the respective surface of each of hollow needle electrodes 302a, 302b, and 302c may include forming the layer of CNTs on surface of respective bevel-shaped tip 312 of each of hollow needle electrodes 302a, 302b, and 302c.

In an exemplary implementation, forming the layer of CNTs on the respective surface of each of hollow needle electrodes 302a, 302b, and 302c may include growing a plurality of CNTs on the respective surface of each of hollow needle electrodes 302a, 302b, and 302c and welding the grown plurality of CNTs to the respective surface of each of hollow needle electrodes 302a, 302b, and 302c.

In an exemplary implementation, growing the plurality of CNTs on the respective surface of each of hollow needle electrodes 302a, 302b, and 302c may include exposing hollow needle electrodes 302a, 302b, and 302c to oxygen plasma and immersing whole or parts of hollow needle electrodes 302a, 302b, and 302c in a solution including dispersed CNTs. In an exemplary implementation, immersing whole or parts of hollow needle electrodes 302a, 302b, and 302c in the solution including dispersed CNTs may further include sonicating the solution to form a highly dispersed solution of CNTs. In an exemplary implementation, immersing whole or parts of hollow needle electrodes 302a, 302b, and 302c in the solution including dispersed CNTs may further include preparing the solution by dispersing CNTs in a mixture of ethanol and deionized (DI) water. In an exemplary implementation, sonicating the solution may include sonicating the solution utilizing at least one of a sonicator horn and a sonication bath. In an exemplary implementation, sonicating the solution may be done at a sonication power in a range between 100 W and 150 W. In an exemplary implementation, growing the plurality of CNTs on the respective surface of each of hollow needle electrodes 302a, 302b, and 302c may include adhesion of CNTs to hollow needle electrodes 302a, 302b, and 302c right after immersion of hollow needle electrodes 302a, 302b, and 302c in the solution.

In an exemplary implementation, forming the layer of CNTs on the respective surface of each of hollow needle electrodes 302a, 302b, and 302c may further include forming a firm attachment between the grown CNTs and the respective surface of each of hollow needle electrodes 302a, 302b, and 302c by welding or soldering the grown CNTs to the respective surface of each of hollow needle electrodes 302a, 302b, and 302c. In an exemplary implementation, welding the grown plurality of CNTs to the respective surface of each of hollow needle electrodes 302a, 302b, and 302c may include welding the grown plurality of CNTs to the respective surface of each of hollow needle electrodes 302a, 302b, and 302c by laser-assisted welding (nano-welding) of CNTs to an outer respective surface of each of hollow needle electrodes 302a, 302b, and 302c utilizing a laser-assisted welding (nano-welding) process. In an exemplary implementation, laser-assisted nano-welding of CNTs to the respective surface of each of hollow needle electrodes 302a, 302b, and 302c may include breaking C-C bonds of CNTs and forming carbon-metal (C-M) bonds; thereby, resulting in decreasing electrical contact resistance between CNTs and metallic surface of hollow needle electrodes 302, for example, platinum hollow needle electrodes.

In an exemplary implementation, laser-assisted welding of CNTs on the respective surface of each of three hollow needle electrodes 302a, 302b, and 302c may include placing hollow needle electrodes 302a, 302b, and 302c with grown CNTs thereon in a sealed container, filling the sealed container with a noble gas, for example, Argon (Ar), and irradiating laser to hollow needle electrodes 302a, 302b, and 302c with grown CNTs thereon. Irradiating the laser leads to rise temperature within the sealed container up to several thousand degrees Celsius (° C.), for example, more than about 1000° C. In an exemplary embodiment, the noble gas may be configured to be a neutralizer agent preventing oxidization of CNTs and/or hollow needle electrodes 302a, 302b, and 302c due to high temperatures caused by laser irradiation inside the sealed container. In an exemplary implementation, irradiating laser to hollow needle electrodes 302a, 302b, and 302c with grown CNTs thereon may include irradiating a continuous-wave laser with a wavelength of about 1064 nm. In an exemplary implementation, irradiating laser to hollow needle electrodes 302a, 302b, and 302c with grown CNTs thereon may be done within a time period in a range between 5 seconds and 10 seconds utilizing a laser irradiator device.

FIG. 3B shows a schematic view of exemplary laser-welded MWCNTs onto an exemplary surface 330 of an exemplary hollow needle electrode of three hollow needle electrodes 302, consistent with one or more exemplary embodiments of the present disclosure. In an exemplary embodiment, laser-assisted nano-welded MWCNTs may be firmly attached on exemplary surface 330 of a cylindrical platinum electrode as an example of each of hollow needle electrodes 302a, 302b, and 302c. Schematic view of FIG. 3B depicts laser-induced structural defects and open-ended carbon atoms of an exemplary MWCNT 340, which may be formed by a strong carbon-metal bonding at laser-irradiated areas.

Moreover, step 406 may include electrically insulating the respective surface of each of hollow needle electrodes 302a, 302b, and 302c except a respective distal portion of each of hollow needle electrodes 302a, 302b, and 302c. In an exemplary implementation, electrically insulating the respective surface of each of hollow needle electrodes 302a, 302b, and 302c except the respective distal portion of each of hollow needle electrodes 302a, 302b, and 302c may include coating a layer of an electrical insulating material, for example, a layer of plastic, around parts of surface of each of hollow needle electrodes 302a, 302b, and 302c except a respective length of each of hollow needle electrodes 302a, 302b, and 302c configured to form exemplary lipid sensitive part 306 for each hollow needle electrode. In an exemplary embodiment, a respective length of each of hollow needle electrodes 302a, 302b, and 302c configured to form exemplary lipid sensitive part 306 may include a length of about 5 mm to 15 mm of respective distal portion of each of hollow needle electrodes 302a, 302b, and 302c.

In an exemplary implementation, three hollow needle electrodes 302a, 302b, and 302c with respective exemplary lipid sensitive part 306 for each hollow needle electrode may be formed by conducting steps 402, 404, and 406 of exemplary process 400 described hereinabove. Additionally, exemplary process 400 may further include connecting a respective first end of each electrical connector line of three electrical connector lines 316 to a respective proximal end of each of hollow needle electrodes 302a, 302b, and 302c (step 408). In an exemplary implementation, three electrical connector lines 316 may be configured to be connected to an electrochemical stimulator-analyzer device; thereby, connecting three hollow needle electrodes 302 to the electrochemical stimulator-analyzer device. In an exemplary embodiment, a respective second end of each electrical connector line of three electrical connector lines 316 may be configured to be connected to the electrochemical stimulator-analyzer device.

Furthermore, step 410 may include placing three hollow needle electrodes 302 with respective electrical connector lines 316 inside exemplary handle 304. In an exemplary implementation, placing three hollow needle electrodes 302 with respective electrical connector lines 316 inside exemplary handle 304 may include forming three holes at exemplary locations 324, 326, and 328 of exemplary bottom surface 308 and passing hollow needle electrodes 302a, 302b, and 302c through respective three holes at locations 324, 326, and 328, so that exemplary lipid sensitive part 306 of each hollow needle electrode of three hollow needle electrodes 302 may be placed outside of bottom surface 308. In an exemplary implementation, passing hollow needle electrodes 302a, 302b, and 302c through respective three holes at locations 324, 326, and 328 may include attaching a respective cross outer surface of hollow needle electrodes 302a, 302b, and 302c to a respective internal cross surface of three holes at locations 324, 326, and 328.

In an exemplary embodiment, handle 304 may be configured to facilitate inserting a respective lipid sensitive part 306 of each of three hollow needle electrodes 302 into an exemplary LN. In an exemplary embodiment, respective proximal ends of three hollow needle electrodes 302 may be placed inside handle 304. In an exemplary embodiment, the respective second end of each electrical connector line of three electrical connector lines 316 may be placed outside an exemplary second end 322 of handle 304.

In an exemplary implementation, forming three holes at exemplary locations 324, 326, and 328 of exemplary bottom surface 308 may include forming three circular openings with a respective diameter of each opening equal to a diameter of each hollow needle electrode of three hollow needle electrodes 302. In an exemplary embodiment, a diameter of each circular opening may be in a range between about 0.1 mm and 1 mm. In an exemplary embodiment, a diameter of each circular opening may be about 0.5 mm.

In an exemplary implementation, forming three holes at exemplary locations 324, 326, and 328 of exemplary bottom surface 308 may be done taking into account an average size of an exemplary LN, since all three respective lipid sensitive parts of three hollow needle electrodes 302 should be inserted into an exemplary LN to conduct an electrical measurement, for example, recording an electrochemical impedance spectroscopy (EIS) of lipid content in an exemplary LN. For example, an average size of adult female standard breast (sentinel or axillary) LN is less than about 1 cm in diameter, and a diameter of cancer involved LNs are mostly less than about 5 cm. Accordingly, an exemplary distance between each two respective electrode of three hollow needle electrodes 302 may be designed to lead to an optimum current density between three hollow needle electrodes 302 being generated. In an exemplary implementation, forming three holes at exemplary locations 324, 326, and 328 of exemplary bottom surface 308 may include forming three openings at exemplary locations 324, 326, and 328 with a triangular pattern at exemplary bottom surface 308. In an exemplary embodiment, each two openings at exemplary locations 324, 326, and 328 may be formed apart from each other by a distance between about 1 mm and about 5 mm. In an exemplary embodiment, an exemplary distance between each two respective electrode of three hollow needle electrodes 302 may be set equal to about 3 mm resulting in a current density of about 0.1 μA/mm² to about 10 μA/mm² in different frequencies of EIS recording, which may be an optimum situation for a dielectric spectroscopy approach.

Moreover, step 412 may include forming an opening 318 at a location of body of handle 304 above a location of respective proximal ends of three hollow needle electrodes 302. In an exemplary embodiment, exemplary opening 318 may be configured to inject a solution, for example, a biocompatible electrolyte solution through opening 318 into at least one hollow needle electrode of three hollow needle electrodes 302.

Referring to FIG. 1B, exemplary method 100A may further include generating a reference R_{CT} value utilizing exemplary fabricated LDP 300 (step 104), measuring fatty acid oxidation (FAO) in a suspected lymph node (LN), utilizing exemplary fabricated LDP 300, by measuring a R_{CT} of the suspected LN (step 110), and detecting, utilizing one or more processors, a cancerous status of the suspected LN based on the measured R_{CT} value (step 120).

In detail, step 104 may include generating a reference R_{CT} value. In an exemplary implementation, generating the reference R_{CT} value may include measuring a first set of R_{CT} values associated with a plurality of healthy lymph nodes (LNs), measuring a second set of R_{CT} values associated with a plurality of cancer involved LNs, and determining the reference R_{CT} value by determining a R_{CT} value at a border line between the first set of R_{CT} values and the second set of R_{CT} values. In an exemplary implementation, measuring the first set of R_{CT} values associated with the plurality of healthy lymph nodes (LNs) and measuring the second set of R_{CT} values associated with the plurality of cancer involved LNs may be done utilizing exemplary LDP 300.

In an exemplary implementation, determining the reference R_{CT} value by determining a R_{CT} value at a border line between the first set of R_{CT} values and the second set of R_{CT} values may include assigning a cut-off value between the first set of R_{CT} values and the second set of R_{CT} values to the reference R_{CT} value. In an exemplary embodiment, the reference R_{CT} value may be more than a maximum value of R_{CT} values associated with the plurality of cancer involved LNs while being less than a minimum of the first set of R_{CT} values associated with the plurality of healthy LNs. In an exemplary embodiment, the reference R_{CT} value may be determined equal to 110 kΩ.

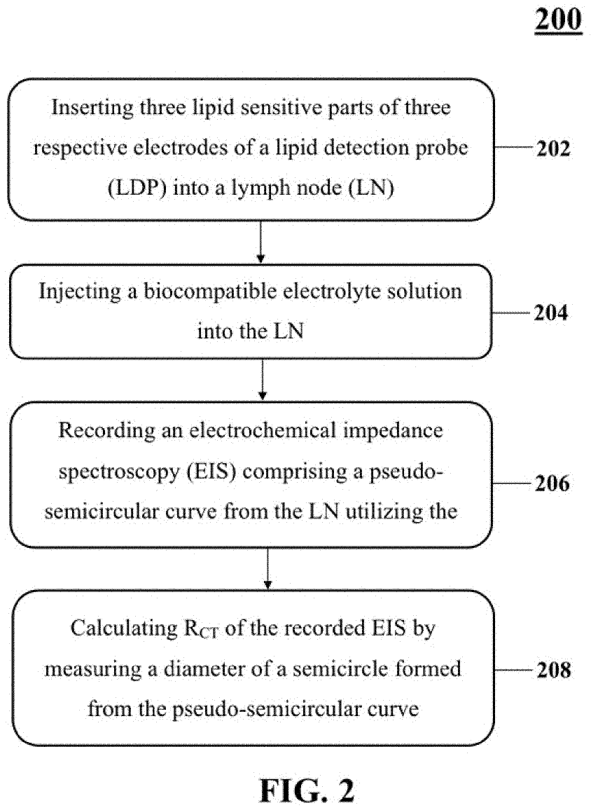

Furthermore, step 110 may include measuring FAO in a suspected LN by measuring a R_{CT} value associated with the LN utilizing exemplary LDP 300. In an exemplary implementation, each step of measuring each of R_{CT} values associated with each LN of the plurality of cancer involved LNs, the plurality of healthy LNs, and the suspected LN for exemplary steps similar to steps 104 and/or 110 may be done via an exemplary method 200 shown in FIG. 2. Exemplary method 200 may include an exemplary method for measuring FAO in a LN via measuring a R_{CT} associated with an exemplary FAO mechanism in the LN.

FIG. 2 shows an exemplary method 200 for measuring FAO in a LN via measuring a R_{CT} value of the LN, consistent with one or more exemplary embodiments of the present disclosure. In an exemplary implementation, exemplary method 200 may be conducted in-vitro for measuring FAO in a LN dissected from a living body. In an exemplary implementation, exemplary method 200 may be conducted in-vivo for measuring FAO in a LN within a living body.

In an exemplary implementation, exemplary LDP 300 may be utilized for conducting method 200. In an exemplary implementation referring to FIG. 2, exemplary method 200 may include inserting exemplary three lipid sensitive parts 306 of three respective electrodes 302 of LDP 300 into a LN (step 202), injecting a biocompatible electrolyte solution into the LN (step 204), recording an electrochemical impedance spectroscopy (EIS), including a pseudo-semicircular curve, from the LN utilizing LDP 300 and an electrochemical stimulator-analyzer device (step 206), and calculating R_{CT} of the recorded EIS by measuring a diameter of a semicircle curve formed from the pseudo-semicircular curve (step 208). In an exemplary embodiment, the LN may include at least one of an exemplary suspected LN that is to be evaluated to check whether it is healthy or cancer involved, a LN of the plurality of cancer involved LNs, a LN of the plurality of healthy LNs, and combinations thereof. In an exemplary embodiment, a cancer involved LN may refer to an LN which contains cancerous cells migrated to the LN.

In detail, step 202 may include inserting exemplary three lipid sensitive parts 306 of three respective electrodes 302 of LDP 300 into an exemplary LN. In an exemplary implementation, inserting exemplary three lipid sensitive parts 306 of three respective electrodes 302 of LDP 300 into an exemplary LN may include inserting each respective distal end 312 of each of hollow needle electrodes 302a, 302b, and 302c into at least one of a LN in a living body in case of an in-vivo implementation and a dissected LN from a living body in case of an in-vitro implementation.

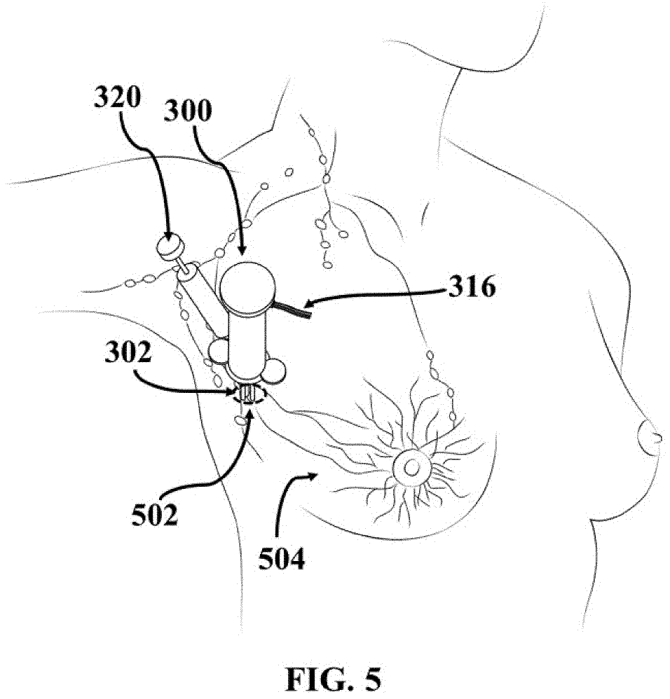

FIG. 5 shows an exemplary schematic implementation of in-vivo inserting exemplary three lipid sensitive parts 306 of three respective electrodes 302 of LDP 300 into an exemplary LN 502, consistent with one or more exemplary embodiments of the present disclosure. In an exemplary embodiment, LN 502 may be a suspected LN to be cancerous that is adjacent to an exemplary breast tissue 504 that is cancer involved.

Furthermore, step 204 may include injecting a biocompatible electrolyte solution into an exemplary LN, for example, LN 502 shown in FIG. 5. In an exemplary implementation, step 204 may include increasing electrical conductivity inside an exemplary LN by injecting the biocompatible electrolyte solution into an exemplary LN; thereby, resulting in facilitating transfer of electrons between an exemplary working electrode and an exemplary counter electrode of three hollow needle electrodes 302. In other words, the biocompatible electrolyte solution may act as an electrical carrier solution within an exemplary LN between an exemplary working electrode and an exemplary counter electrode. In an exemplary implementation, referring to FIG. 5, injecting the biocompatible electrolyte solution into an exemplary LN may include injecting the biocompatible electrolyte solution into LN 502 utilizing syringe 320. In an exemplary implementation, injecting the biocompatible electrolyte solution into LN 502 may include filling syringe 320 with the biocompatible electrolyte solution and injecting the biocompatible electrolyte solution into LN 502. In an exemplary implementation, injecting the biocompatible electrolyte solution into an exemplary LN may include injecting the biocompatible electrolyte solution through at least one of three hollow needle electrodes 302 of LDP 300 by injecting the biocompatible electrolyte solution into a respective proximal end of the at least one of three hollow needle electrodes 302 utilizing syringe 320.

In an exemplary embodiment, the biocompatible electrolyte solution may include a biocompatible and electrically conductive solution containing one or more metal ions. In an exemplary embodiment, the biocompatible electrolyte solution may include an injectable solution of iron ions. In an exemplary embodiment, the solution of iron ions may include a colloidal solution of ferric carboxymaltose complex. In an exemplary embodiment, the colloidal solution of ferric carboxymaltose complex may include a colloidal solution of ferric carboxymaltose complex with a concentration in a range between about 1 mg/ml and 50 mg/ml.

Moreover, step 206 may include recording an EIS from an exemplary LN, for example, LN 502, utilizing LDP 300 and an electrochemical stimulator-analyzer device. In an exemplary implementation, recording the EIS from exemplary LN 502 may include connecting exemplary LDP 300 to an electrochemical stimulator-analyzer device via three electrical connector lines 316, applying an AC voltage between about 5 mV and about 10 mV by sweeping a frequency range between about 0.01 Hz and about 100 kHz utilizing the electrochemical stimulator-analyzer device connected to LDP 300, measuring a set of electrical impedance of LN 502 respective to the swept frequency range utilizing the electrochemical stimulator-analyzer device connected to LDP 300, and plotting a respective set of imaginary part of impedance (Z″ (Ω)) of the set of electrical impedance versus a respective set of real part of impedance (Z′ (Ω)) of the set of electrical impedance.

In an exemplary embodiment, the frequency range may include a plurality of frequency values between about 0.01 Hz and about 100 kHz. In an exemplary embodiment, the electrochemical stimulator-analyzer device may include a potentiostat device.

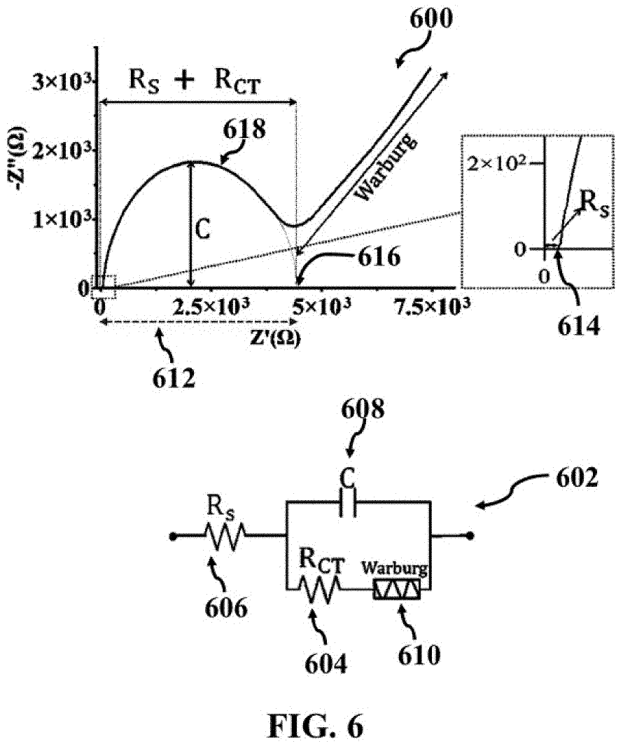

In an exemplary implementation, plotting the respective set of imaginary part of impedance (Z″ (Ω)) of the set of electrical impedance versus the respective set of real part of impedance (Z′ (Ω)) of the set of electrical impedance may be conducted utilizing one or more processors. In an exemplary implementation, plotting the respective set of imaginary part of impedance (Z″ (Ω)) of the set of electrical impedance versus the respective set of real part of impedance (Z′ (Ω)) of the set of electrical impedance may include plotting a Nyquist plot with a pseudo-semicircular curve shape from an exemplary LN 502. In an exemplary embodiment, an exemplary plot of the set of electrical impedance (an exemplary EIS plot) may be a Nyquist plot which may be analyzed by a corresponding equivalent circuit (named a Randles circuit). FIG. 6 shows a schematic view of exemplary EIS plot 600 recorded from an exemplary LN 502, consistent with one or more exemplary embodiments of the present disclosure. Exemplary EIS plot 600 may include an exemplary Nyquist diagram with a—pseudo-semicircular curve shape including a set of recorded imaginary part of impedance (Z″ (Ω)) versus a set of recorded real part of impedance (Z′ (Ω)). In an exemplary embodiment, exemplary EIS plot 600 may be a Nyquist diagram that may be electrically simulated or fitted with exemplary respective equivalent circuit 602 (an exemplary Randles circuit). In an exemplary embodiment, exemplary circuit 602 may be used to analyze and quantitatively fitting electrical behavior of exemplary LN 502 measured and recorded by EIS plot 600. In an exemplary embodiment, exemplary circuit 602 may include two corresponding electrical resistances through an electrical current path between an exemplary working electrode and an exemplary counter electrode of three hollow needle electrodes 302. In an exemplary embodiment, the two corresponding electrical resistances may include an exemplary charge transfer resistance (R_{CT}) 604 of an exemplary LN 502 and a solution resistance (R_{S}) 606. In an exemplary embodiment, R_{S} 606 may be an electrical resistance of an exemplary fluid within exemplary LN 502. In an exemplary implementation, exemplary R_{S} 606 may be minimized by injecting the biocompatible electrolyte solution into LN 502 in step 204. In an exemplary embodiment, exemplary circuit 602 may further include exemplary double layer capacitance C 608 and exemplary diffusion element (Warburg) 610.

Furthermore, step 208 may include calculating R_{CT} of the recorded EIS by measuring a diameter of a semicircle curve associated with the pseudo-semicircular curve. In an exemplary implementation, calculating R_{CT} of the recorded EIS may be done utilizing one or more processors. Referring to FIG. 6, exemplary EIS plot 600 may include pseudo-semicircular curve 618. In an exemplary embodiment, pseudo-semicircular curve 618 may start from exemplary first intersection point 614 between EIS plot 600 and Z′ (Ω) axis. In an exemplary implementation, calculating R_{CT} of the recorded EIS may include measuring a value of first intersection point 614 on Z′ (Ω) axis, forming a semicircle curve from pseudo-semicircular curve 618, and measuring a diameter of the formed semicircle curve. In an exemplary implementation, forming the semicircle curve may include adding a complementary sector to pseudo-semicircular curve 618 to form a semicircle curve. In an exemplary implementation, forming the semicircle curve may include continuing pseudo-semicircular curve 618 towards Z′ (Ω) axis that may include generating exemplary second intersection point 616 between EIS plot 600 and Z′ (Ω) axis. In an exemplary implementation, measuring the diameter of the formed semicircle curve may include calculating exemplary distance 612 between exemplary first intersection point 614 and exemplary second intersection point 616.

Referring again to FIGS. 1A and 1B, step 120 may include detecting a cancerous status of the suspected LN based on the measured R_{CT} value. In an exemplary implementation, detecting the cancerous status of the suspected LN based on the measured R_{CT} value may be done utilizing one or more processors. In an exemplary implementation, detecting the cancerous status of the suspected LN based on the measured R_{CT} value may include comparing the measured R_{CT} value with a reference R_{CT} value, and detecting the cancerous status of the suspected LN is healthy or cancer involved. In an exemplary implementation, comparing the measured R_{CT} value with the reference R_{CT} value may be done utilizing one or more processors.

In an exemplary implementation, detecting the cancerous status of the suspected LN may further include detecting the suspected LN is a cancer involved LN if the measured R_{CT} is less than the reference R_{CT} value or detecting the suspected LN is a healthy LN if the measured R_{CT} is more than the reference R_{CT} value. In an exemplary implementation, detecting the cancerous status of the suspected LN may include comparing, utilizing one or more processors, the measured R_{CT} with a reference R_{CT} value of about 110 kΩ and detecting, utilizing one or more processors, the suspected LN is a cancer involved LN if the measured R_{CT} is less than about 110 kΩ or detecting, utilizing one or more processors, the suspected LN is a healthy LN if the measured R_{CT} is less than about 110 kΩ.

### EXAMPLE 1

### Preparing Lipid Detection Probe (LDP)

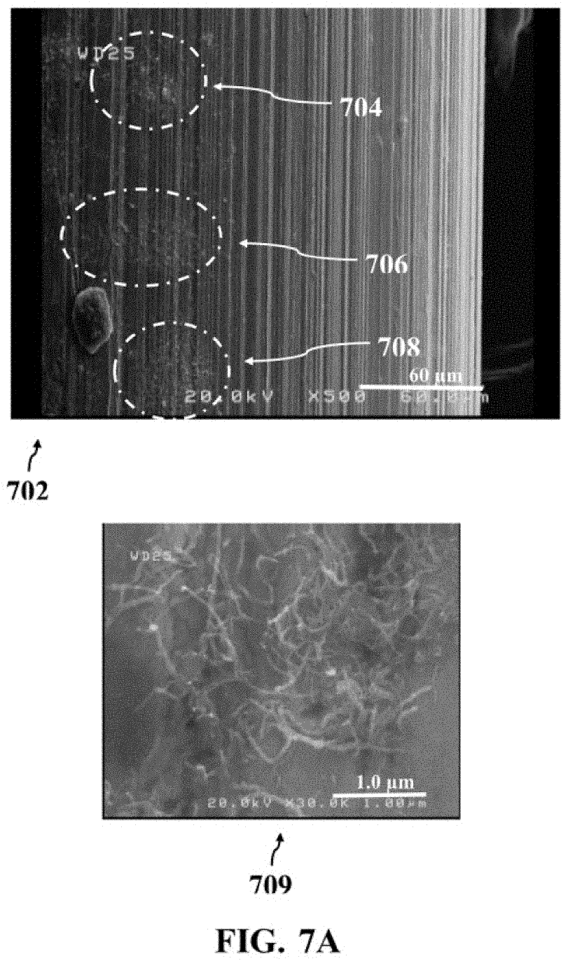

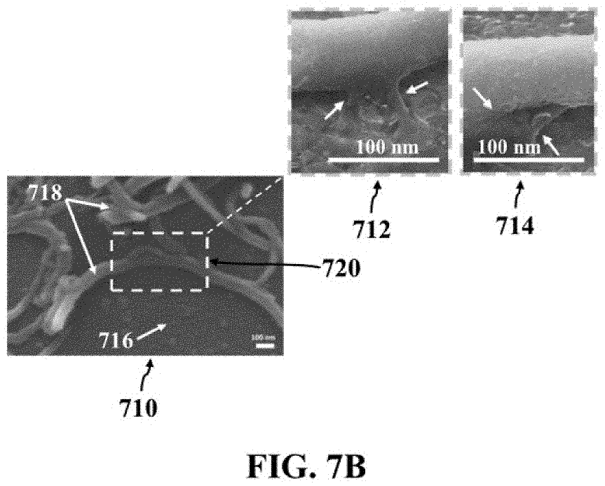

In this example, an exemplary LDP similar to LDP 300 was prepared for utilizing in exemplary methods 100 and 200 for diagnosis of cancer involved LNs. Three platinum, 0.5 mm thickness, hollow needle electrodes were formed thin with a bevel-shaped tip using wiring and cylinder formation methods, and mechanochemical method. Platinum needles were exposed to oxygen plasma and immersed in a dispersed CNT solution to weld CNTs on surface of needles. The dispersed CNT solution was prepared by dispersing 0.1 mg of multi-walled carbon nanotubes (MWCNTs) in ethanol:deionized (DI) water (3:1, V:V). To obtain well-dispersed CNTs, the solution was sonicated using a sonicator horn at a power of 100 W for about 30 minutes in a cycle of 7:3 seconds (On:Off). FIG. 7A shows scanning electron microscopy (SEM) images 702 of exemplary arrays 704, 706, and 708 of VAMWCNTs attached on an exemplary electrode of an exemplary prepared LDP and a magnified image 709 of exemplary attached VAMWCNTs array 708, consistent with one or more exemplary embodiments of the present disclosure. As may be seen, SEM images proved adhesion of CNTs to Pt needles right after Pt needles immersion.

Afterwards, CNT-Pt needles were placed in a sealed container purged by noble gas (Ar) and laser irradiation. The laser irradiation was conducted using a 1064 nm wavelength laser. FIG. 7B shows field emission-scanning electron microscopy (FE-SEM) images 710, 712, and 714 of exemplary array of VAMWCNTs welded on exemplary three-integrated electrodes of an exemplary prepared LDP, consistent with one or more exemplary embodiments of the present disclosure. FE-SEM image 710 shows exemplary MWCNTs 718 soldered onto surface of platinum substrate 716. Exemplary part 720 of image 710 is magnified in images 712 and 714. White arrows in images 712 and 714 clearly show exemplary formed welding sites of exemplary MWCNTs 718 on exemplary platinum needle 716. Scale bars in images 710, 712, and 714 show a length of 100 nm.

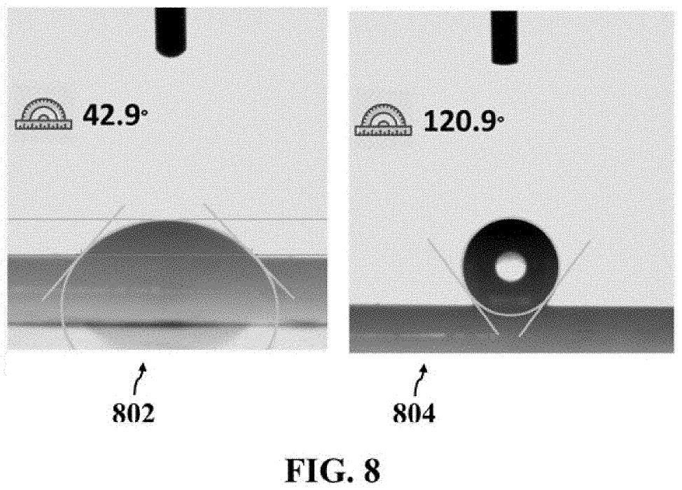

Furthermore, wettability of prepared electrodes was investigated through contact angle measurement. Droplet of fresh Dulbecco's Modified Eagle's medium (DMEM) cell culture media (a solution with no lipid) has been used to measure the contact angle. FIG. 8 shows contact angles of an exemplary bare platinum electrode (image 802) and an exemplary MWCNT welded platinum electrode (image 804), consistent with one or more exemplary embodiments of the present disclosure. A lipid-free droplet of cell culture media, the DMEM droplet, was formed with a contact angle of 42.9° on bare Pt electrode, while a contact angle of the DMEM droplet on MWCNT welded Pt electrode was 120.9° which illustrates hydrophobic behavior of MWCNT welded Pt electrodes. The measured contact angles prove MWCNT welded electrodes' hydrophobic behavior, which results in better interaction of MWCNT welded electrodes with a lipidic media.

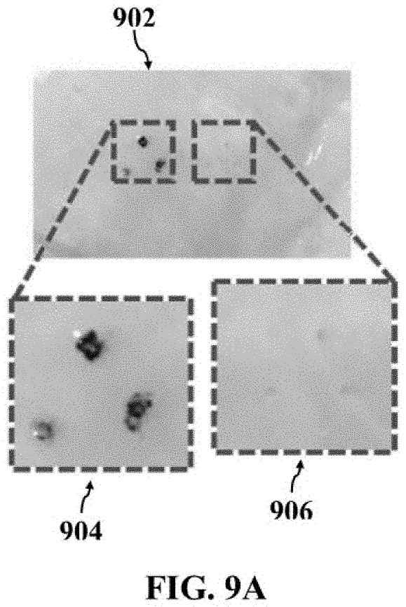

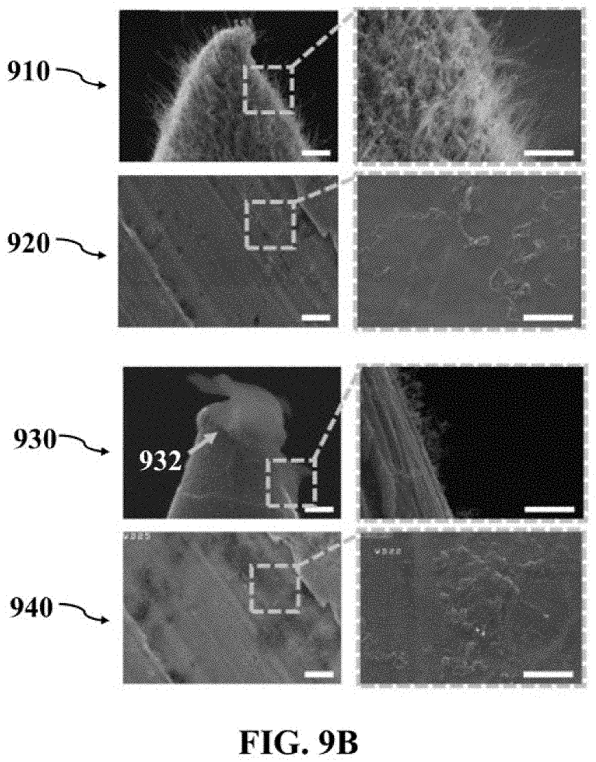

To compare welded CNT electrodes (named as welded CNT Pt electrode (WCPE)) with electrodes that CNTs had been only grown thereon utilizing a direct-current plasma enhanced chemical vapor deposition (DC-PECVD) technique in a DC-PECVD reactor (named as grown CNT on Pt electrode (GCPE)), exemplary electrodes were penetrated into a euthanized animal tissue and a footprint image was recorded. FIG. 9A shows residue footprint (image 902) after penetration of an exemplary prepared CNT welded platinum electrode (magnified in image 906) compared to an exemplary platinum electrode with grown CNTs thereon (magnified in image 904), consistent with one or more exemplary embodiments of the present disclosure. As it is evident, penetration trace of exemplary GCPE is easily detectable. Remnants are peeled-off CNTs from exemplary GCPE. While almost there is no residue of CNTs remaining in exemplary tissue for exemplary WCPE. Hence, attachment of exemplary WCPE is far better than exemplary GCPE, which results in no residue in an exemplary examined tissue after needle entrance. Exemplary penetrated electrodes were gently rinsed with deionized (DI) water to remove tissue contents on them. Afterward, exemplary penetrated electrodes were analyzed by FE-SEM. FIG. 9B shows FE-SEM images of penetrated platinum electrode with grown CNTs thereon before (image 910) and after (image 930) penetration to an exemplary animal tissue in comparison with penetrated CNT welded platinum electrode before (image 920) and after (image 940) penetration to an exemplary animal tissue, consistent with one or more exemplary embodiments of the present disclosure. Scale bars in all images of FIG. 9B indicates a length of 1 μm. FE-SEM images show a difference in C-Pt adhesion between WCPE and. These images clearly depict that CNTs of GCPE were wiped out after penetration. FE-SEM images of exemplary WCPE demonstrate that CNTs still stuck to electrodes even after penetration. Exemplary blur black shadows 932 may be hydrophobic contents of exemplary tissue (firmly may be lipids) adsorbed to welded CNTs.

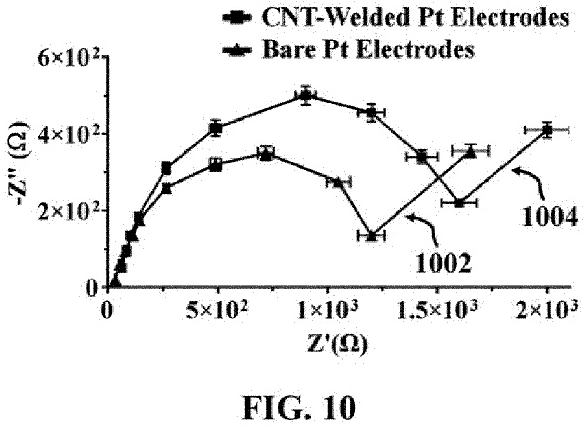

For analyzing effect of exemplary MWCNTs welded on exemplary platinum needle electrodes, a comparative graph of EIS results between exemplary bare Pt electrodes and Pt electrodes decorated by welded CNTs in Dulbecco's Modified Eagle Medium (DMEM) with 10% Fetal Bovine Serum (FBS), as a lipidic solution, was recorded. FIG. 10 shows EIS analysis results of exemplary bare platinum electrodes (diagram 1002) versus MWCNT welded platinum electrodes (diagram 1004), consistent with one or more exemplary embodiments of the present disclosure. Fresh cell culture media (DMEM+10% FBS) has been tested as a standard lipidic solution. Exemplary CNT welded electrode shows higher R_{CT} value due to better interaction of electrode with lipid contents of lipidic media.

A length of each of three exemplary MWCNT welded Pt electrodes, fabricated similar to exemplary hollow needle electrodes 302a, 302b, and 302c, were insulated except a distal end portion which remained non-insulated for insertion into an exemplary LN. A plastic material was covered around surface of each of three exemplary MWCNT welded Pt electrodes except a respective distal end portion. Three exemplary MWCNT welded Pt electrodes were attached and fixed at the end of a handle with a distance of 3 mm from each other. Three respective electrodes include a set of a working electrode (WE), a reference electrode (RE), and a counter electrode (CE) for that may be utilized in EIS recording and measurements.

### EXAMPLE 2

### Recording Fatty Acid Content in LNs of Animal Model

Animal model procedures were accomplished in-vivo via an implementation of exemplar method 100 utilizing a miniaturized fabricated LDP similar to exemplary LDP 300. A proper animal model may be critical for mimicking human disease conditions. Hence, adult white strains of New Zealand rabbits with a weight of 2.5 kg to 3 kg were used in this trial. Anesthesia was performed using ketamine 50 mg/kg and xylazine 10 mg/kg intravenous and intramuscular, respectively. Rabbit LNs were used to clarify effect of lipid content in EIS response of LN ambient, and EIS was recorded before and after injection of Tumescent (as a lipolyzing agent) into an exemplary LN of rabbit to simulate lipid consumption by a cancer involved LN. Tumescent is a lipolysis solution that includes lidocaine and epinephrine, which increases phosphorylation status of perilipin in adipocytes and subjects them to lipolysis. Tumescent also has a local anesthesia effect on skin and its subcutaneous tissue. Tumescent solution contains 500 mg of lidocaine, 0.5-1 mg of epinephrine, and 10 mEq of sodium bicarbonate in 1 L of 0.9% normal saline. Before EIS recording of lymph nodes, 250 μl of a solution of ferric carboxymaltose complex (2 mg/ml) as an electrically conductive carrier solution had been directly injected into LNs of an exemplary animal model.

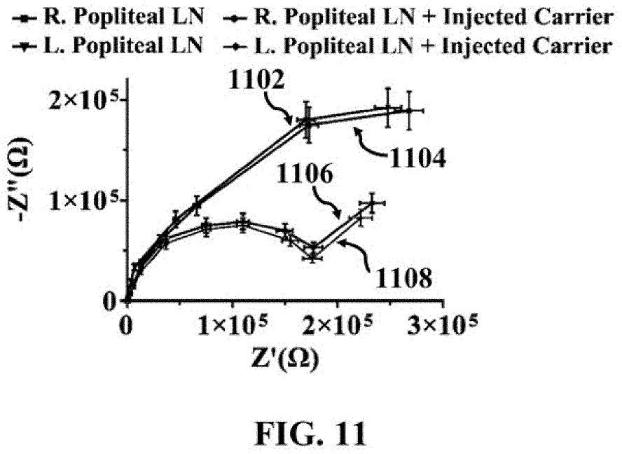

FIG. 11 shows EIS recorded diagrams of rabbit right and left popliteal LNs with and without injecting electrically conductive carrier solution, consistent with one or more exemplary embodiments of the present disclosure. Diagram 1102 shows EIS recorded diagram of rabbit left popliteal LN without injecting electrically conductive carrier solution, diagram 1104 shows EIS recorded diagram of rabbit right popliteal LN without injecting electrically conductive carrier solution, diagram 1106 shows EIS recorded diagram of rabbit right popliteal LN with injecting electrically conductive carrier solution, and diagram 1108 shows EIS recorded diagram of rabbit left popliteal LN with injecting electrically conductive carrier solution. It may be observable that injected carrier turned the EIS curve to a meaningful shape with indicated R_{CT}. It may indicate that an exemplary ionic carrier is necessary for EIS analyses and LN EIS responses did not complete without injecting ionic carrier.

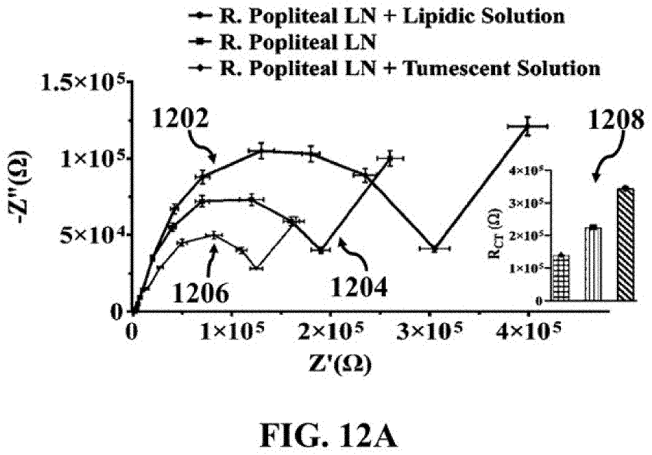

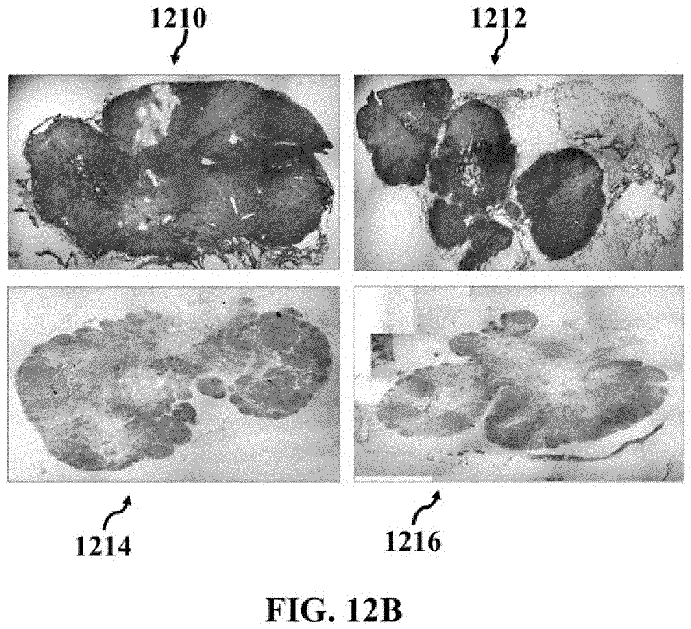

FIG. 12A shows EIS recorded diagrams of right popliteal lymph nodes of rabbit for a LN with an injected lipidic solution (diagram 1202), a normal lymph node (diagram 1204), and a lymph node with injected tumescent solution (diagram 1206) and an inset diagram 1208 of respective R_{CT} of each EIS, consistent with one or more exemplary embodiments of the present disclosure. A drastic reduction in R_{CT} (as a central part of EIS response) from about 300 kΩ towards about 120 kΩ after LNs' interaction with tumescent is observed, which revealed an effect of LN lipid content in EIS responses. R_{CT} magnitude has been reduced by decreasing an amount of lipidic contents of LNs.

Additionally, FIG. 12B shows permanent and frozen Hematoxylin and eosin (H&E) histopathological staining results of normal and tumescent injected popliteal LNs of rabbit, consistent with one or more exemplary embodiments of the present disclosure. In detail, images 1210 and 1212 shows permanent H&E histopathological staining results of normal and tumescent injected popliteal LNs of rabbit, respectively, and images 1214 and 1216 shows frozen H&E histopathological staining results of normal and tumescent injected popliteal LNs of rabbit, respectively. As it is obvious, tumescent solution significantly decreased an amount of lipidic content in lymph nodes, it is why an EIS response of these lymph nodes behaves like materials with lower dielectric compared to normal, and lipid solution injected lymph nodes. Pathological evaluation of normal and lipolyzed LNs indicated that reduced content of lipids is in correlation with decreased R_{CT} in EIS responses of LNs. Image processing results carried out on pathological images demonstrate that normal LNs include high amounts of lipids and tumescent solution significantly reduced the lipidic contents, which correlates with EIS results.

### EXAMPLE 3

### Analyzing Cancerous Status of Human LNs Utilizing Exemplary Fabricated LDP

As exemplary EIS approach in animal model (described hereinabove in Example 2) showed meaningful results on differentiating LNs with various contents of lipids, exemplary method 100, utilizing exemplary LDP 300, was applied on dissected LNs of 41 patients, immediately after dissection. In this regard, exemplary LDP 300 was applied to 122 LNs (including sentinel and non-sentinel) from breast cancer patients, which had been dissected through standard guidelines. Patients undergoing breast cancer surgery were recruited to this analysis. During a breast cancer surgery, manual lymph node dissection was performed. Right after lymph node dissection, lymph nodes have been tested by injecting carriers and penetrating exemplary fabricated electrodes of exemplary fabricated LDP to them. Moreover, LN dissected samples were sent for frozen pathology. Obtained results of permanent pathology were conducted to compare with results obtained from EIS recording according to exemplary method 100. EIS recording procedure was carried out in less than 1 minute. To carry out electrochemical spectroscopy of dielectric components of dissected LNs, 250 μl of a solution of ferric carboxymaltose complex (2 mg/ml) was directly injected into LNs right after dissection. Exemplary EIS responses were recorded using a portable potentiostat by conducting EIS at 10 mV with a frequency range of 100 kHz to 0.01 Hz at 10 points. In order to ensure about Pt electrodes calibration, they tested and calibrated using potassium ferricyanide (K₃Fe(CN)₆) before each sterilization step. In each sterilization step, exemplary fabricated electrodes were sterilized by autoclave. The autoclaved electrodes were transferred to a biological safety container and sealed in a medical-grade sterilized package, including paraformaldehyde tablets. Sterilization step was renewed after 2 weeks of storing.

Table 1 shows measured R_{CT} values of Patients' LNs using an exemplary LDP, responses of EIS measurements by LDP, frozen section, permanent pathology section, and EIS result evaluation in comparison to permanent histopathological diagnosis in 45 SLNs and 77 non-sentinel LNs. A border of normal and cancer involved LNs was detected at about 110 kΩ. True-positive and false-positive results have been specified by comparing EIS results with permanent pathology. Each test was repeated for 5 times with a standard deviation (STD) of about ±5%.

TABLE 1
Measured R_{CT} values of Patients' LNs using an exemplary LDP, responses
of EIS measurements by LDP, frozen section, permanent pathology section,
and EIS result evaluation in comparison to permanent histopathological
diagnosis in 45 SLNs and 77 non-sentinel LNs.

| | | | | | | | Response |
|---|---|---|---|---|---|---|---|
| | | | | | | | Compared |
| Patient | Patient | Type of | R_{CT} (Ω) | Frozen | LDP | Permanent | to |
| 1 | i | Sentinel | 5.85E+04 | + | + | + | TP |
| | ii | Non-sentinel | 9.82E+04 | − | + | + | TP |
| | iii | Non-sentinel | 1.43E+05 | − | − | − | TN |
| 2 | i | Sentinel | 4.00E+05 | − | − | − | TN |
| | ii | Non-sentinel | 4.95E+05 | − | − | − | TN |
| 3 | i | Sentinel | 4.00E+05 | − | − | − | TN |
| | ii | Non-sentinel | 3.76E+05 | − | − | − | TN |
| 4 | i | Sentinel | 1.30E+04 | + | + | + | TP |
| | ii | Sentinel | 1.43E+05 | − | − | − | TN |
| 5 | i | Sentinel | 1.44E+04 | + | + | + | TP |
| | ii | Non-sentinel | 2.52E+04 | + | + | + | TP |
| | iii | Non-sentinel | 4.90E+05 | − | − | − | TN |
| | iv | Non-sentinel | 5.04E+05 | − | − | − | TN |
| 6 | i | Sentinel | 2.55E+04 | + | + | + | TP |
| | ii | Non-sentinel | 1.57E+05 | − | − | − | TN |
| | iii | Non-sentinel | 1.45E+05 | − | − | − | TN |
| 7 | i | Sentinel | 2.15E+04 | + | + | + | TP |
| | ii | Non-sentinel | 3.49E+05 | − | − | − | TN |
| 8 | i | Sentinel | 6.80E+04 | + | + | + | TP |
| | ii | Non-sentinel | 7.40E+04 | + | + | + | TP |
| 9 | i | Sentinel | 5.13E+05 | − | − | − | TN |
| | ii | Non-sentinel | 4.95E+05 | − | − | − | TN |
| 10 | i | Sentinel | 1.60E+05 | − | − | − | TN |
| | ii | Sentinel | 1.85E+05 | − | − | − | TN |
| | iii | Non-sentinel | 1.41E+05 | − | − | − | TN |
| 11 | i | Sentinel | 1.90E+04 | + | + | + | TP |
| | ii | Non-sentinel | 1.40E+04 | + | + | + | TP |
| | iii | Non-sentinel | 1.49E+04 | + | + | + | TP |
| | iv | Non-sentinel | 4.98E+05 | − | − | − | TN |
| 12 | i | Sentinel | 1.34E+04 | + | + | + | TP |
| | ii | Non-sentinel | 1.44E+05 | − | − | − | TN |
| | iii | Non-sentinel | 4.94E+05 | − | − | − | TN |
| | iv | Non-sentinel | 4.99E+05 | − | − | − | TN |
| | v | Non-sentinel | 1.46E+05 | − | − | − | TN |
| 13 | i | Sentinel | 1.33E+04 | + | + | + | TP |
| | ii | Non-sentinel | 1.63E+05 | − | − | − | TN |
| | iii | Non-sentinel | 1.87E+05 | − | − | − | TN |
| | iv | Non-sentinel | 4.88E+05 | − | − | − | TN |
| 14 | i | Sentinel | 1.37E+04 | + | + | + | TP |
| | ii | Non-sentinel | 1.45E+05 | − | − | − | TN |
| | iii | Non-sentinel | 1.65E+05 | − | − | − | TN |
| | iv | Non-sentinel | 1.46E+04 | + | + | + | TP |
| | v | Non-sentinel | 3.96E+05 | − | − | − | TN |
| 15 | i | Sentinel | 1.29E+04 | − | + | − | FP |
| | ii | Sentinel | 1.41E+05 | − | − | − | TN |
| 16 | i | Sentinel | 1.44E+04 | + | + | + | TP |
| | ii | Non-sentinel | 1.42E+05 | − | − | − | TN |
| | iii | Non-sentinel | 6.94E+04 | + | + | + | TP |
| | iv | Non-sentinel | 3.42E+05 | − | − | − | TN |
| 17 | i | Sentinel | 3.62E+04 | + | + | + | TP |
| | ii | Non-sentinel | 1.05E+04 | + | + | + | TP |
| | iii | Non-sentinel | 3.45E+05 | − | − | − | TN |
| | iv | Non-sentinel | 1.44E+05 | − | − | − | TN |
| 18 | i | Sentinel | 5.03E+05 | − | − | − | TN |
| | ii | Non-sentinel | 1.89E+05 | − | − | − | TN |
| 19 | i | Sentinel | 1.92E+04 | + | + | + | TP |
| | ii | Non-sentinel | 3.52E+05 | − | − | − | TN |
| | iii | Non-sentinel | 7.33E+04 | + | + | + | TP |
| | iv | Non-sentinel | 1.40E+05 | − | − | − | TN |
| 20 | i | Sentinel | 4.08E+05 | − | − | − | TN |
| | ii | Non-sentinel | 4.12E+05 | − | − | − | TN |
| 21 | i | Sentinel | 1.38E+05 | − | − | − | TN |
| | ii | Non-sentinel | 1.47E+05 | − | − | − | TN |
| 22 | i | Sentinel | 1.40E+05 | − | − | − | TN |
| | ii | Non-sentinel | 4.04E+05 | − | − | − | TN |
| 23 | i | Sentinel | 1.86E+04 | + | + | + | TP |
| | ii | Non-sentinel | 1.52E+04 | + | + | + | TP |
| | iii | Non-sentinel | 1.40E+05 | − | − | − | TN |
| | iv | Non-sentinel | 1.42E+05 | − | − | − | TN |
| 24 | i | Sentinel | 1.81E+05 | − | − | − | TN |
| | ii | Non-sentinel | 5.08E+05 | − | − | − | TN |
| 25 | i | Sentinel | 1.94E+04 | + | + | + | TP |
| | ii | Non-sentinel | 1.48E+04 | + | + | + | TP |
| | iii | Non-sentinel | 1.44E+05 | − | − | − | TN |
| | iv | Non-sentinel | 5.00E+05 | − | − | − | TN |
| 26 | i | Sentinel | 1.62E+05 | − | − | − | TN |
| | ii | Sentinel | 3.96E+05 | − | − | − | TN |
| 27 | i | Sentinel | 3.80E+05 | − | − | − | TN |
| | ii | Non-sentinel | 1.44E+05 | − | − | − | TN |
| 28 | i | Sentinel | 1.03E+04 | + | + | + | TP |
| | ii | Non-sentinel | 7.47E+04 | + | + | + | TP |
| | iii | Non-sentinel | 1.41E+04 | + | + | + | TP |
| | iv | Non-sentinel | 1.40E+05 | − | − | − | TN |
| 29 | i | Sentinel | 1.00E+03 | + | + | + | TP |
| | ii | Non-sentinel | 9.40E+04 | − | + | + | TP |
| | iii | Non-sentinel | 1.04E+04 | + | + | + | TP |
| 30 | i | Sentinel | 1.31E+04 | + | + | + | TP |
| | ii | Non-sentinel | 2.60E+04 | + | + | + | TP |
| | iii | Non-sentinel | 1.42E+05 | − | − | − | TN |
| | iv | Non-sentinel | 1.38E+05 | − | − | − | TN |
| 31 | i | Sentinel | 9.62E+04 | + | + | + | TP |
| | ii | Non-sentinel | 1.58E+05 | − | − | − | TN |
| | iii | Non-sentinel | 3.87E+05 | − | − | − | TN |
| 32 | i | Sentinel | 1.07E+04 | + | + | + | TP |
| | ii | Non-sentinel | 1.39E+04 | + | + | + | TP |
| | iii | Non-sentinel | 1.47E+05 | − | − | − | TN |
| 33 | i | Sentinel | 2.60E+04 | + | + | + | TP |
| | ii | Non-sentinel | 3.39E+05 | − | − | − | TN |
| | iii | Non-sentinel | 4.89E+04 | + | + | + | TP |
| | iv | Non-sentinel | 3.59E+05 | − | − | − | TN |
| 34 | i | Sentinel | 1.65E+05 | − | − | − | TN |
| | ii | Non-sentinel | 5.13E+05 | − | − | − | TN |
| 35 | i | Sentinel | 3.65E+05 | − | − | − | TN |
| | ii | Non-sentinel | 1.45E+05 | − | − | − | TN |
| 36 | i | Sentinel | 3.55E+04 | + | + | + | TP |
| | ii | Non-sentinel | 7.18E+04 | + | + | + | TP |
| | iii | Non-sentinel | 5.10E+05 | − | − | − | TN |
| | iv | Non-sentinel | 1.47E+05 | − | − | − | TN |
| 37 | i | Sentinel | 1.96E+04 | + | + | + | TP |
| | ii | Non-sentinel | 1.39E+05 | − | − | − | TN |
| | iii | Non-sentinel | 1.34E+04 | + | + | + | TP |
| | iv | Non-sentinel | 1.39E+05 | − | − | − | TN |
| 38 | i | Sentinel | 5.67E+04 | + | + | + | TP |
| | ii | Non-sentinel | 1.48E+04 | + | + | + | TP |
| | iii | Non-sentinel | 1.79E+05 | − | − | − | TN |
| 39 | i | Sentinel | 1.30E+04 | + | + | + | TP |
| | ii | Non-sentinel | 1.45E+05 | − | − | − | TN |
| | iii | Non-sentinel | 3.88E+05 | − | − | − | TN |
| 40 | i | Sentinel | 4.80E+05 | − | − | − | TN |
| | ii | Non-sentinel | 1.47E+05 | − | − | − | TN |
| 41 | i | Sentinel | 2.60E+04 | + | + | + | TP |
| | ii | Non-sentinel | 3.39E+05 | − | − | − | TN |

Results presented in Table 1 show a meaningful correlation between measured R_{CT} of recorded EIS responses and pathological diagnosis about cancer cell involvement of LNs. Pathological diagnosis was done by H&E and Pan Cytokeratin, PCK, which are based immune histochemistry assays. Significantly, lower R_{CT} was recorded for cancerous LNs in comparison with normal ones.

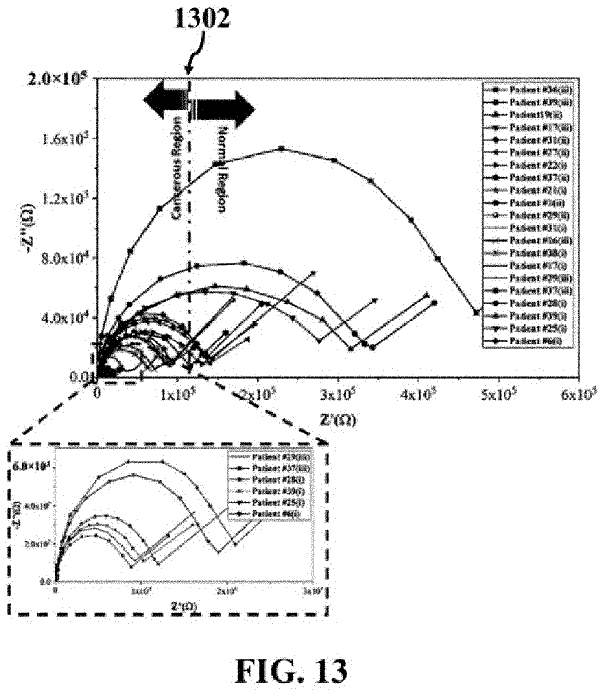

FIG. 13 shows EIS recorded responses for a plurality of exemplary sentinel LNs of tested patients, consistent with one or more exemplary embodiments of the present disclosure. Results depict a meaningful border (R_{CT}) 1302 at about 110 kΩ to differentiate cancerous and normal lymph nodes. The lowest measured R_{CT} for normal LNs is about 1.41×10⁵Ω and the highest value for involved LNs is about 9.4×10⁴Ω; thus, a median (with similar distance from the lowest and highest values) may be an appropriate R_{CT} reference value 1302 as a cut-off or borderline between normal and cancerous LN zones, which may be determined to be equal to 110 kΩ.

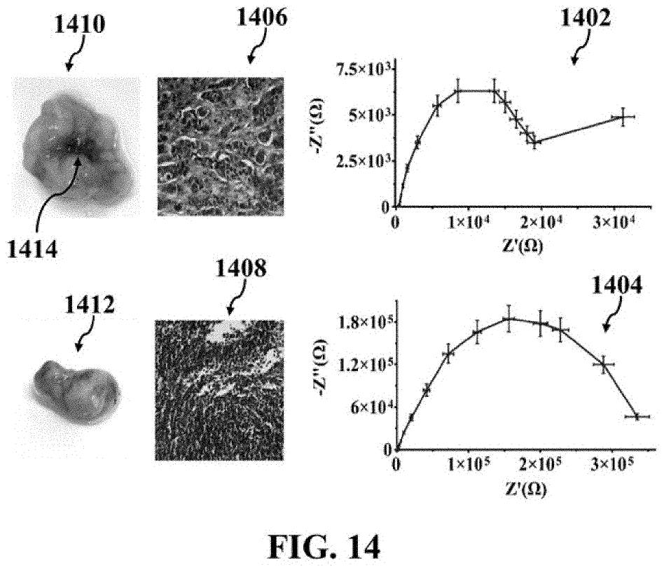

FIG. 14 shows comparative EIS recorded responses 1402 and 1404, pathological analyzes results 1406 and 1408, and optical images 1410 and 1412, respectively for an exemplary cancer involved LN No. 1 of patient No. 7 and an exemplary normal LN No. 3 of patient No. 17 of Table 1, consistent with one or more exemplary embodiments of the present disclosure. Optical image 1410 clearly illustrates an exemplary involved LN with black spot 1414 with a lower amount of R_{CT} of 2.15×10⁴Ω measured from EIS 1402 in comparison with R_{CT} of 3.45×10⁵Ω measured from EIS 1404. Besides the hardness, black spots in LNs are the most common diagnostic factors for the surgeons during breast cancer surgery.

As presented in Table 1, only the first lymph node of patient No. 15 was falsely scored positive by EIS analysis. This case had undergone neoadjuvant chemotherapy with complete remission, but she lived with cancer for a while. Hence, cancer cells in invaded LNs by cancer cells had enough time to deplete LN lipids for their metabolism. So, LNs depleted from lipid due to cancer cells FAO metabolism before conducting a chemotherapy. As a consequence, this result might be due to a depletion of lipids that occurred from cancerous cells before chemotherapy.

It should be noted that the only way to quantify invading tumor cells in LNs is pathologists' diagnosis. Therefore, it will be very practitioner-dependent. Also, some other variables affect the pathological results. For instance, number of slides provided from tissue sections, evaluated area of the slides, and even number of slides that evaluated could directly affect a pathologists' diagnosis. Diagnosis of pathologists for defining a cancer involvement percentage were conducted for dissected LNs, and a mean value of their diagnosis was set as an involvement percentage to cancer. By plotting R_{CT} versus involvement percentage, a relative relation between cancer involvement percentage and R_{CT} magnitude was extrapolated.

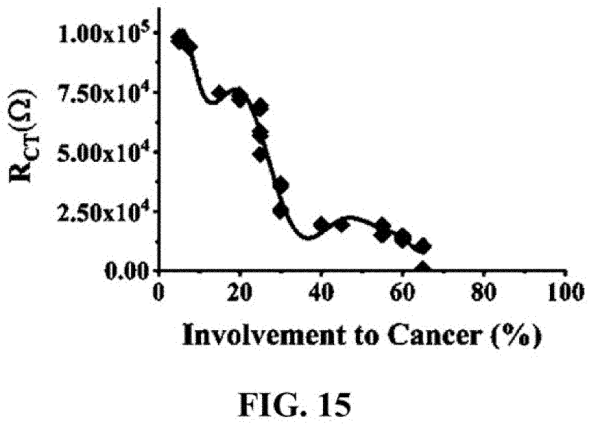

FIG. 15 shows R_{CT} values versus LNs' involvement percentage graph representing a relation between EIS results and cancer progression in the LNs, consistent with one or more exemplary embodiments of the present disclosure. It should be noted that in 41 patients' LNs analysis utilizing exemplary LDP 300 (122 LNs), all types or extensive amounts of involvement were not detected. For instance, there is not a case of matted lymph node, which presents total involvement. Results demonstrates two drastic changes of R_{CT} in two involvement ranges of 5-15% and 20-40% LN involvement to cancer, which means up to about 40% of cancerous involvement in LNs would result in a significant increase of LNs' conductivity due to consumption of lipid dielectric materials by cancer cells during FAO metabolism. A mild reduction in R_{CT} after involvement to further percent of cancer cells was observed which may be due to a much lower distribution of lipidic contents.

By EIS investigation of diagnosed LNs (according to pathology gold standard), a primary warning factor for probability of lymph node involvement based on R_{CT} was defined. EIS values obtained for clear (normal or healthy) and cancer involved LNs may be helpful to have an early estimation from the cancerous state of a LN before its dissection and sending to frozen pathology during surgery. The obtained results show real-time ability of such nano electro biochemical methodology based on a strong biological pathway, FAO, in in-vivo findings of LN cancer involvement before a LN dissection.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for detecting cancerous status of a suspected lymph node (LN) to be cancerous, comprising:
forming a lipid detection probe (LDP) by forming three lipid sensitive parts at three respective distal ends of three hollow needle electrodes by laser-assisted welding of a layer of carbon nanotubes (CNTs) onto surface of the respective distal ends of three platinum hollow needles;
connecting a proximal end of each respective hollow needle electrode to an electrochemical stimulator-analyzer device;
inserting the three lipid sensitive parts into the suspected LN;
injecting a biocompatible electrolyte solution into the LN through a respective proximal end of at least one hollow needle electrode of the three hollow needle electrodes;
measuring fatty acid oxidation (FAO) in the suspected LN by measuring a charge transfer resistance (R_{CT}) associated with the suspected LN, comprising:
recording an electrochemical impedance spectroscopy (EIS) from the suspected LN utilizing the LDP and the electrochemical stimulator-analyzer device, the recorded EIS comprising a pseudo-semicircular-shaped curve; and
calculating R_{CT} of the recorded EIS utilizing one or more processors, comprising:
forming a semicircle curve by complementing the pseudo-semicircular-shaped curve; and
measuring a diameter of the semicircle curve; and
detecting, utilizing one or more processors, a cancerous status of the suspected LN, comprising:
detecting the suspected LN being cancer involved responsive to the measured R_{CT} being less than 110 kΩ; or
detecting the suspected LN being healthy responsive to the measured R_{CT} being more than 110 k∜.

2. A method for detecting cancerous status of a suspected lymph node (LN) to be cancerous, comprising:
measuring fatty acid oxidation (FAO) in the suspected LN by measuring a charge transfer resistance (R_{CT}) associated with the suspected LN; and
detecting, utilizing one or more processors, a cancerous status of the suspected LN, comprising:
detecting the suspected LN being cancer involved responsive to the measured R_{CT} being less than a reference R_{CT} value; or
detecting the suspected LN being healthy responsive to the measured R_{CT} being more than the reference R_{CT} value.

3. The method of claim 2, wherein detecting the cancerous status of the suspected LN comprises:
comparing, utilizing one or more processors, the measured R_{CT} with a reference R_{CT} value of 110 kΩ; and
detecting the cancerous status of the suspected LN, comprising:
detecting, utilizing one or more processors, the suspected LN being cancer involved responsive to the measured R_{CT} being less than 110 kΩ; or
detecting, utilizing one or more processors, the suspected LN being healthy responsive to the measured R_{CT} being less than 110 kΩ.

4. The method of claim 2, further comprising generating the reference R_{CT} value, comprising:
measuring a first set of R_{CT} values associated with a plurality of healthy lymph nodes (LNs);
measuring a second set of R_{CT} values associated with a plurality of cancer involved LNs; and
determining the reference R_{CT} value by determining a R_{CT} value at a border line magnitude between the first set of R_{CT} values and the second set of R_{CT} values.

5. The method of claim 4, wherein measuring each of R_{CT} values associated with each LN of the plurality of cancer involved LNs, the plurality of healthy LNs, and the suspected LN comprises:
inserting three lipid sensitive parts of three respective electrodes of a lipid detection probe (LDP) into a LN, the LN comprising one of the suspected LN, a LN of the plurality of cancer involved LNs, and a LN of the plurality of healthy LNs, each respective electrode comprising a hollow needle electrode, each lipid sensitive part comprising a distal end of each respective hollow needle electrode coated with a layer of lipophilic electrically conductive nanostructures, the layer of lipophilic electrically conductive nanostructures comprising a layer of carbon nanotubes (CNTs);
increasing electrical conductivity inside the LN by injecting a biocompatible electrolyte solution into the LN;
recording an electrochemical impedance spectroscopy (EIS) from the LN utilizing the LDP, the recorded EIS comprising a pseudo-semicircular-shaped curve; and
calculating R_{CT} of the recorded EIS, utilizing one or more processors, by measuring a diameter of a semicircle associated with the pseudo-semicircular-shaped curve.

6. The method of claim 5, wherein inserting three lipid sensitive parts of three respective electrodes of the LDP into the LN comprises inserting each respective distal end of each electrode of the three electrodes into at least one of a LN in a living body and a dissected LN from a living body.

7. The method of claim 5, wherein recording the EIS from the LN comprises:
connecting the LDP to an electrochemical stimulator-analyzer device;
applying an AC voltage between 5 mV and 10 mV by sweeping a frequency range, the frequency range comprising a plurality of frequency values between 0.01 Hz and 100 kHz;
measuring a set of electrical impedance of the LN respective to the swept frequency range; and
forming the pseudo-semicircular-shaped curve by plotting a respective set of imaginary part of impedance (Z′ (Ω)) of the set of electrical impedance versus a respective set of real part of impedance (Z′ (Ω)) of the set of electrical impedance.

8. The method of claim 7, wherein calculating the R_{CT} of the recorded EIS comprises:
measuring a first intersection point of the pseudo-semicircular-shaped curve with Z′ (Ω) axis;
generating a second intersection point between the pseudo-semicircular-shaped curve and Z′ (Ω) axis by adding a complementary sector to the pseudo-semicircular-shaped curve to form a semicircle; and
measuring a distance between the first intersection point and the second intersection point.

9. The method of claim 5, wherein injecting the biocompatible electrolyte solution into the LN comprises injecting the biocompatible electrolyte solution through at least one of the three electrodes of the LDP by injecting the biocompatible electrolyte solution into a respective proximal end of the at least one of the three electrodes utilizing a syringe.

10. The method of claim 5, wherein injecting the biocompatible electrolyte solution into the LN comprises injecting a biocompatible and electrically conductive solution comprising metal ions into the LN.

11. The method of claim 10, wherein injecting the biocompatible electrolyte solution into the LN comprises injecting a solution of iron ions into the LN, the solution of iron ions comprising a colloidal solution of ferric carboxymaltose complex.

12. The method of claim 5, further comprising fabricating the LDP, comprising:
forming the three hollow needle electrodes with the three respective lipid sensitive parts, comprising:
forming a bevel-shaped tip at a respective distal end of each of the three hollow needle electrodes, the bevel-shaped tip configured to facilitate a non-invasive insertion of each of the three hollow needle electrodes into a LN; and
forming a layer of CNTs on the respective distal end of each of the three hollow needle electrodes, the three hollow needle electrodes comprising a working electrode, a counter electrode, and a reference electrode;
connecting respective first ends of three electrical connector lines to respective proximal ends of the three hollow needle electrodes, a respective second end of each electrical connector line being configured to be connected to an electrochemical stimulator-analyzer device;
placing the three hollow needle electrodes with the respective electrical connector lines inside a handle, the handle comprising a hollow cylinder with a bottom surface at a first end of the hollow cylinder, the handle configured to facilitate inserting the three hollow needle electrodes into the LN, the respective distal end of each hollow needle electrode being placed outside the bottom surface; and
forming an opening at a location of the handle above a location of respective proximal ends of the three hollow needle electrodes, the opening being configured to inject the biocompatible electrolyte solution there through into at least one of the three hollow needle electrodes.

13. The method of claim 12, wherein forming the layer of CNTs on the respective distal end of each of the three hollow needle electrodes comprises:
growing a layer of CNTs on each respective distal end of each of the three hollow needle electrodes; and
welding the grown layer of CNTs to the respective distal end of each of the three hollow needle electrodes utilizing a laser welding process.

14. The method of claim 13, wherein growing the layer of CNTs on each respective distal end of each of the three hollow needle electrodes comprises:
preparing a solution of dispersed CNTs by dispersing CNTs in a mixture of ethanol and deionized water;
immersing respective distal end of each of the three hollow needle electrodes in the solution of dispersed CNTs; and
sonicating the solution of dispersed CNTs.

15. The method of claim 13, wherein welding the grown layer of CNTs to the respective distal end of each of the three hollow needle electrodes comprises:
placing the three hollow needle electrodes with grown CNTs on the respective distal ends in a sealed container;
filling the sealed container with a noble gas; and
irradiating continuous-wave laser with a wavelength of 1024 nm to the three hollow needle electrodes with grown CNTs on the respective distal ends in the presence of the noble gas.

16. The method of claim 12, wherein forming the three hollow needle electrodes with the three respective lipid sensitive parts further comprises electrically insulating parts of surface of each hollow needle electrode by covering a layer of an electrical insulating material around the surface of each hollow needle electrode except a surface of the respective distal end of each hollow needle electrode.

17. The method of claim 12, wherein placing the three hollow needle electrodes with the respective electrical connector lines inside the handle comprises:
forming three openings with a triangular pattern at the bottom surface, each two openings being apart from each other by a distance between 1 mm and 5 mm; and
placing the three hollow needle electrodes with the respective electrical connector lines inside the handle by passing each of the three hollow needle electrodes through a respective opening of the formed three openings,
wherein three respective lipid sensitive parts are placed outside the bottom surface.

18. The method of claim 17, wherein:
the respective proximal end of each of the three hollow needle electrodes is placed inside the handle; and
a second end of each respective electrical connector line of the three electrical connector lines is placed outside a second end of the handle.

19. The method of claim 2, wherein measuring FAO in the suspected LN and detecting the cancerous status of the suspected LN are done in a time period of less than one minute.
